(19) United States
(12) Patent Application Publication
(10) Pub. No.: US 2008/0243094 A1
(43) Pub. Date: Oct. 2, 2008

(54) BATHTUB APPARATUS, THERAPEUTIC BATHTUB APPARATUS, BATHING WATER AND THERAPEUTIC BATHING WATER

(76) Inventors: Kazuyuki YAMASAKI, Hiroshima (JP); Takahide Miyamoto, Hiroshima (JP); Kazumi Chuhjoh, Kagawa (JP); Masaki Kataoka, Hiroshima (JP)

Correspondence Address: MARK D. SARALINO ( SHARP );RENNER, OTTO, BOISSELLE & SKLAR, LLP, 1621 EUCLID AVENUE, 19TH FLOOR, CLEVELAND, OH, 44115, US

(21) Appl. No.: 12/056,435

(22) Filed: Mar. 27, 2008

(30) Foreign Application Priority Data
Mar. 30, 2007 (JP) 2007-93215

(51) Int. Cl. A61H 33/02; A61M 35/00

(52) U.S. Cl. 604/289; 4/541.1; 601/154; 977/904

(57) ABSTRACT

A bathtub apparatus is provided, including a nanobubble and/or nanosized medical component generating section to combine at least either of nanobubbles or a nanosized medical component with bath water from a bathtub and to circulate the bath water to the bathtub.

This Nonprovisional application claims priority under 35 U.S.C. §119(a) on Patent Application No. 2007-93215 filed in Japan on Mar. 30, 2007, the entire contents of which are hereby incorporated by reference.

### BACKGROUND OF THE INVENTION

1. Field of the Invention

The present invention relates to a bathtub apparatus for treating various illnesses and maintaining health through bathing as well as a bathing method using the bathtub apparatus, in particular to the bathing apparatus for effectively generating carbon dioxide nanobubbles and air nanobubbles as gas as well as a nanosized medical component as liquid and for combining the nanobubbles or nanosized medical component with bathtub water, in which the nanobubbles or medical component contained in the bathtub water can be easily absorbed into the user's skin when a user bathes with this bathtub water; the medical component is then taken in capillary vessels and circulated around the body, thereby enabling a therapeutic effect for various illnesses and maintaining health. The present invention also relates to a therapeutic bathtub apparatus using the bathtub apparatus; bathing water used therein; and therapeutic bathing water containing the bathing water.

2. Description of the Related Art

Traditionally, carbon dioxide and medical components are used for treating various illnesses and maintaining health, which are a medical field and a health field. For example, a carbonate spring is medically known to increase blood flow, and in Europe, especially in Germany, carbon dioxide or a medical component contained in bathtub water to be absorbed through the skin is known to increase blood flow. A hot spring that contains about 1000 ppm of carbon dioxide, a typical example of a carbonated spring, exists in Europe, and particularly in Germany, and such a carbonated spring has been used for various medical treatments and health maintenance for a long time. In addition, a medical component is provided to a patient as a pharmaceutical from a hospital or a pharmacy under a physician's prescription. Further, treatment and hygiene that utilize herbal medicine traditionally exists although a medical component absorbed through the skin in such treatment or hygiene is limited. For example, amur cork or red-berried elder as herbal medicine is applied at an infected part of the body, so that a medical component from such herbal medicine can be absorbed, rehmannia root is applied to a bruise for treatment, and Artemisia or strawberry geranium is applied to an infection for treatment. Further, iris leaf, citrus, angelica root, chamomilla, cnidium rhizome, citrus unshiu peel, or ginseng is put into a bathtub for various treatments and maintenance of health to promote blood flow.

In Japan, it is said that there are eight million diabetic patients, who suffer from a blood flow problem, and there are even a case that leads to artificial dialysis at a kidney due to complication and a case of a leg amputation because of gangrene due to a blood flow problem near the end of a foot. Accordingly, it is considered effective to take a bath in a carbonate spring capable of increasing blood flow as a remedial measure for a blood flow problem.

However, such a hot spring containing about 1000 ppm of carbon dioxide does not exist in Japan, and therefore, a hot spring that is capable of providing medical treatment for various kinds of illnesses does not exist in Japan. A bathtub water generating apparatus that generates bathtub water containing artificially generated and highly-concentrated carbon dioxide of 1000 ppm or higher (artificial carbonate spring) is available from MRC Home Products Co., Ltd. This bathtub water generating apparatus dissolves carbon dioxide into bathtub water using a semipermeable membrane or a multilayer composition hollow fiber membrane that only allows gas to pass and not water.

As a side note, this nanobubble technique is being watched with keen interest for applications in various fields, such as a health purpose and a beauty treatment.

For example, Reference 1 discloses a utilization method and an apparatus for generating nanobubbles in respect to washing treatment. The conventional technique disclosed in Reference 1 utilizes nanobubble characteristics, such as low buoyancy, high surface area, high surface activity, interfacial activity due to the generation of a partially high-pressured electric field and realization of electrostatic polarization, and bactericidal action. More specifically, Reference 1 discloses that various things can be washed with high effectiveness and low environmental load due to the absorption feature of stain component, high speed washing feature of the substance surface, and bactericidal feature resulted from the correlation of the nanobubbles characteristics described above.

In addition, for example, Reference 2 discloses a method for generating nano-foams as another conventional technique with regard to sewage disposal. The conventional technique disclosed in Reference 2 includes, with regard to liquid, (1) a step of dissolving and gasifying a portion of a liquid, (2) a step of applying a ultra sound wave into a liquid, or (3) a step of dissolving and gasifying a portion of a liquid and a step of applying a ultra sound wave into the liquid.

In addition, for example, Reference 3 discloses a disposal apparatus for waste fluid using ozone microbubbles as still another conventional technique. Reference 3 discloses the provision of ozone gas generated by an ozone generating apparatus and waste fluid taken from underneath a disposal tub in a microbubble generating apparatus via a pressure pump, and, in turn, the sending of generated ozone microbubbles into waste fluid through an opening of a gas discharge pipe.

In addition, for example, Reference 4 discloses a method that applies carbon dioxide microbubbles as still another conventional technique. Reference 4 discloses a conventional technique, in which a carbon dioxide container and a pressure-reducing valve are positioned at an air intake section of a microbubble generating section, for which the dissolution efficiency reaches close to 100% of a theoretical amount by providing pressured carbon dioxide and stable flow rate under a specific condition, allowing of economical performance thanks to a significant reduction in the amount of gas used compared to the previous technique; the corresponding apparatus is smaller in size.

Non-patent Reference 1 describes that IGF-1 (Insulin-like growth factor-1), which transfers a signal similar to insulin that adjusts blood glucose level, lowering blood sugar content, is important for diabetes treatment, and that IGF-1 is further applicable for protein degradation control, reduction of blood pressure, remedy for heart function, remedy for hyperlipidemia, increase of cognitive function, antidepressant, prevention of Alzheimer's disease, skin care, hair growth, healing of wound, anti-inflammation, immune function activation due to activation of natural killer cells.

Reference 1: Japanese Laid-Open Publication No. 2004-121962

Reference 2: Japanese Laid-Open Publication No. 2003-334548

Reference 3: Japanese Laid-Open Publication No. 2004-321959

Reference 4: Japanese Laid-Open Publication No. 2006-320675

Non-patent Reference 1: Prof. Kenji Okajima et al, at medical department of Nagoya City University, “Application and possibility of microbubbles for medical practice”, Clean Technology, January 2007, Japan Industrial Publishing, Co., ltd.

### SUMMARY OF THE INVENTION

In the conventional bathtub water generating apparatus of MRC Home Products Co., Ltd. described above, since carbon dioxide is not used as carbon dioxide micro-nanobubbles or carbon dioxide nanobubbles, it is considered that absorption of carbon dioxide through the skin is insubstantial and therefore, there is little promotion of health, health maintenance effect, or therapeutic effect in such an apparatus.

A system which generates micro-nanobubbles and combines it with bathtub water so as to increase the blood flow of a subject while the subject baths the bathtub water is conceivable as a conventional remedy method for a human leg or foot with blood flow problem. However, a number of spiral flowing micro-nanobubble generators are required in order to demonstrate its effectiveness with respect to increase of blood flow. More specifically, ten spiral flow micro-nanobubble generators are provided in order to demonstrate such an effect. Because of the sheer number of required generators with this spiral flow method, there are significant issues regarding space and cost. Accordingly, this system is not realistic.

Further, References 1-4 described above are related to washing and sewage treatment, and not to a bathtub apparatus and a bathing method for therapy and health. These conventional techniques have the following problems.

References 1-4 described above do not disclose the fact that a medical component is nanosized so that it is absorbed through the skin, taken in capillary vessels and circulated around the body, allowing the demonstration of the medicinal action of the medical component. References 1-4 do not disclose the use of either a medical component tub; a nano-liquid generator that is configured with a liquid-liquid mixture circulating pump having a micro-liquid generating section, a liquid shearing section and a needle valve; or a nano-liquid discharge section in order to generate a nanosized medical component.

Further, References 1-4 described above do not disclose that bathtub water with high discharge pressure combined with carbon dioxide nanobubbles and a nanosized medical component is generated and introduced into a bathtub, and that, when a user bathes in this bathtub water, a nanosized carbon dioxide nanobubbles or nanosized medical component contained in the bathtub water is absorbed into the user's skin, taken in capillary vessels and circulated around the body, thereby treating various illnesses utilizing the blood flow increasing effect of the nanosized carbon dioxide and the medicinal action of the nanosized medical component.

Further, References 1-4 described above do not disclose a configuration of a therapeutic bathtub apparatus that is a combination of a system for generating carbon dioxide nanobubbles from carbon dioxide which is generated from a liquefied carbon dioxide cylinder to be absorbed through the skin to increase blood flow, and a system for generating a nanosized medical component to demonstrate a medicinal action of the medical component in order to obtain a combined effect for treatment.

Further, References 1-4 described above do not disclose how to generate a nanosized medical component with one of various medical components for various illnesses or the combination thereof, so that blood flow is increased and the nanosized medical component is absorbed throughout the human body and is taken in capillary vessels to go around the body, practicing treatments for various illnesses effectively.

As described above, it is conventional to take medication internally or by injection. However, when medication is taken internally, a portion of it is decomposed in a digestive organ, the liver or the kidneys, and therefore, not all of the medication will reach its action site. In addition, when taking a medication internally, it takes a long time for the medication to reach its active site. Further, the aging of patients progresses with the aging of the population, and blood flow of such patients will not be as smooth as when they were young, therefore medication taken internally or by injection may not reach its action site smoothly.

The present invention is provided to solve the conventional problems described above more simply. Carbon dioxide or a medical component that is barely absorbed through the skin is generated by a nanobubble generator or nano-liquid generator as carbon dioxide nanobubbles or a nanosized medical component, and the carbon dioxide nanobubbles or nanosized medical component is combined with bathtub water. When a user bathes in this bathtub water, the carbon dioxide nanobubbles or nanosized medical component contained in the bathtub water is absorbed into the user's skin effectively, taken in capillary vessels and circulated around the body with blood, thereby achieving the objective of the present invention, which is to provide a bathtub apparatus that can provide a therapeutic effect for various illnesses and an effect of maintenance of health. The present invention also includes a therapeutic bathtub apparatus using the bathtub apparatus, bathing water used therein and therapeutic bathing water using the bathing water.

A bathtub apparatus according to the present invention is provided, which includes a nanobubble and/or nanosized medical component generating section to combine at least either of nanobubbles or a nanosized medical component with bath water from a bathtub and to circulate the bath water to the bathtub, thereby achieving the objective described above.

Preferably, in a bathtub apparatus according to the present invention, the nanobubble and/or nanosized medical component generating section includes a gas-liquid mixture circulating pump for mixing and shearing bathtub water from the bathtub and gas from outside by a microbubble generating section to produce cloudy water full of microbubbles; and a gas-liquid shearing section for shearing microbubbles provided from the gas-liquid mixture circulating pump to generate nanobubbles.

Still preferably, in a bathtub apparatus according to the present invention, the nanobubble and/or nanosized medical component generating section includes a gas-liquid mixture circulating pump for mixing and shearing bathtub water from the bathtub and gas from outside by a microbubble generating section to produce cloudy water full of microbubbles; and a high speed rotating section, which is positioned inside the bathtub, for shearing and pulverizing microbubbles provided from the gas-liquid mixture circulating pump with difference between rotating speeds at a front and back of a discharge opening to generate nanobubbles.

Still preferably, in a bathtub apparatus according to the present invention, the nanobubble and/or nanosized medical component generating section includes a liquid-liquid mixture circulating pump for mixing and shearing bathtub water from the bathtub and a medical component liquid from outside by a micro-liquid generating section to produce cloudy water of micro-liquid; and a liquid shearing section for shearing a micro-liquid provided from the liquid-liquid mixture circulating pump to generate a nano-liquid.

Still preferably, in a bathtub apparatus according to the present invention, the nanobubble and/or nanosized medical component generating section includes a liquid-liquid mixture circulating pump for mixing and shearing bathtub water from the bathtub and a medical component liquid from outside by a micro-liquid generating section to produce cloudy water of micro-liquid; and a high speed rotating section, which is positioned inside the bathtub, for shearing and pulverizing a micro-liquid provided from the liquid-liquid mixture circulating pump with difference between rotating speeds at a front and back of a discharge opening to generate a nano-liquid.

Still preferably, in a bathtub apparatus according to the present invention, the nanobubble and/or nanosized medical component generating section includes a gas-liquid/liquid-liquid mixture circulating pump for mixing and shearing bathtub water from the bathtub and gas or a medical component liquid from outside by a microbubble/micro-liquid generating section to produce cloudy water full of microbubbles or a micro-liquid; and a gas-liquid/liquid-liquid shearing section for shearing microbubbles or a micro-liquid provided from the gas-liquid/liquid-liquid mixture circulating pump to generate nanobubbles or a nano-liquid.

Still preferably, in a bathtub apparatus according to the present invention, the nanobubble and/or nanosized medical component generating section includes a gas-liquid/liquid-liquid mixture circulating pump for mixing and shearing bathtub water from the bathtub and gas or a medical component liquid from outside by a microbubble/micro-liquid generating section to produce cloudy water full of microbubbles or a micro-liquid; and a high speed rotating section, which is positioned inside the bathtub, for shearing and pulverizing microbubbles or a micro-liquid provided from the gas-liquid/liquid-liquid mixture circulating pump with difference between rotating speeds at a front and back of a discharge opening to generate nanobubbles or a nano-liquid.

Still preferably, in a bathtub apparatus according to the present invention, the nanobubble and/or nanosized medical component generating section includes a gas-liquid mixture circulating pump for mixing and shearing bathtub water from the bathtub and gas from outside by a microbubble generating section to produce cloudy water full of microbubbles; a gas-liquid shearing section for shearing the microbubbles provided from the gas-liquid mixture circulating pump to generate nanobubbles; a liquid-liquid mixture circulating pump for mixing and shearing bathtub water from the bathtub and a medical component liquid from outside by a micro-liquid generating section to produce cloudy water of a micro-liquid; and a liquid shearing section for shearing a micro-liquid provided from the liquid-liquid mixture circulating pump to generate a nano-liquid.

Still preferably, in a bathtub apparatus according to the present invention, the nanobubble and/or nanosized medical component generating section includes a gas-liquid mixture circulating pump for mixing and shearing bathtub water from the bathtub and gas from outside by a microbubble generating section to produce cloudy water full of microbubbles; a first high speed rotating section, which is positioned inside the bathtub, for shearing and pulverizing microbubbles provided from the gas-liquid mixture circulating pump with difference between rotating speeds at a front and back of a discharge opening to generate nanobubbles; a liquid-liquid mixture circulating pump for mixing and shearing bathtub water from the bathtub and a medical component liquid from outside by a micro-liquid generating section to produce cloudy water of micro-liquid; and a second high speed shearing section, which is positioned inside the bathtub, for shearing and pulverizing a micro-liquid provided from the liquid-liquid mixture circulating pump with difference between rotating speeds at a front and back of a discharge opening to generate a nano-liquid.

Still preferably, in a bathtub apparatus according to the present invention, the nanobubble and/or nanosized medical component generating section includes a gas-liquid mixture circulating pump for mixing and shearing bathtub water from the bathtub and gas from outside by a microbubble generating section to produce cloudy water full of microbubbles; and a gas-liquid shearing section for shearing microbubbles provided from the gas-liquid mixture circulating pump to generate nanobubbles; a liquid-liquid mixture circulating pump for mixing and shearing bathtub water from the bathtub and a medical component liquid from outside by a micro-liquid generating section to produce cloudy water of micro-liquid; and a second high speed rotating section, which is positioned inside the bathtub, for shearing and pulverizing a micro-liquid provided from the liquid-liquid mixture circulating pump with difference between rotating speeds at a front and back of a discharge opening to generate a nano-liquid.

Still preferably, in a bathtub apparatus according to the present invention, the nanobubble and/or nanosized medical component generating section includes a gas-liquid mixture circulating pump for mixing and shearing bathtub water from the bathtub and gas from outside by a microbubble generating section to produce cloudy water full of microbubbles; a first high speed rotating section, which is positioned inside the bathtub, for shearing and pulverizing microbubbles provided from the gas-liquid mixture circulating pump with difference between rotating speeds at a front and back of a discharge opening to generate nanobubbles; a liquid-liquid mixture circulating pump for mixing and shearing bathtub water from the bathtub and a medical component liquid from outside by a micro-liquid generating section to produce cloudy water of micro-liquid; and a liquid shearing section for shearing a micro-liquid provided from the liquid-liquid mixture circulating pump to generate a nano-liquid.

Still preferably, in a bathtub apparatus according to the present invention, the gas is provided for the microbubble generating section via a needle valve.

Still preferably, in a bathtub apparatus according to the present invention, the nanobubbles are at least either of carbon dioxide nanobubbles or air nanobubbles.

Still preferably, in a bathtub apparatus according to the present invention, carbon dioxide as the gas is provided for the microbubble generating section from a liquefied carbon dioxide cylinder via a pressure-reducing valve and a needle valve.

Still preferably, in a bathtub apparatus according to the present invention, air as the gas is provided for the microbubble generating section via a valve.

Still preferably, in a bathtub apparatus according to the present invention, a plurality of nanobubble discharging sections, for which the nanobubbles are provided, are provided inside the bathtub, and a valve, whose degree of opening is independently adjustable, is provided for each of the plurality of the nanobubble discharging sections.

Still preferably, in a bathtub apparatus according to the present invention, a plurality of the high speed rotating sections are provided inside the bathtub, and a valve, whose degree of opening is independently adjustable, is provided for each of the plurality of the high speed rotating sections.

Still preferably, in a bathtub apparatus according to the present invention, a dissolved carbon dioxide analyzer is provided inside the bathtub and a signal input terminal of a dissolved carbon dioxide controller is electrically connected to a signal output terminal of the dissolved carbon dioxide analyzer, and the signal output terminal of the dissolved carbon dioxide controller is electrically connected to a control terminal of a needle valve for which carbon dioxide is provided, and a degree of opening of the needle valve is controlled by the dissolved carbon dioxide controller according to a concentration of dissolved carbon dioxide measured by the dissolved carbon dioxide analyzer, so that the amount of carbon dioxide introduced into the microbubble generating section from the needle valve is adjusted to a predetermined value.

Still preferably, a bathtub apparatus according to the present invention further includes a medical component tub that retains the medical component liquid and a medical component tub pump that is capable of sucking a medical component liquid from the medical component tub and providing the medical component liquid to the micro-liquid generating section.

Still preferably, a bathtub apparatus according to the present invention further includes a needle valve that is capable of controlling the amount of the medical component liquid between the medical component tub pump and the micro-liquid generating section.

Still preferably, in a bathtub apparatus according to the present invention, the medical component tub is filled with an herbal medicine and a medical component extracted from the herbal medicine is mixed with bathing water for use.

Still preferably, in a bathtub apparatus according to the present invention, one of iris leaf, citrus, angelica root, chamomilla, cnidium rhizome, citrus unshiu peel, ginseng, or two or more of the combination thereof is selected as the herbal medicine and fills the medical component tub.

Still preferably, in a bathtub apparatus according to the present invention, a heater, a thermometer and a temperature controller that is electrically connected to the thermometer are provided for the medical component tub, and the temperature controller is electrically connected to the heater, so that a temperature inside the medical component tub is controlled using the heater to a predetermined temperature by the temperature controller according to the temperature inside the medical component tub measured by the thermometer.

A therapeutic bathtub apparatus uses the bathtub apparatus according to the present invention for treatment of various illnesses, thereby achieving the objective described above.

Bathing water is provided, in which either of nanobubbles or a nanosized medical component is combined with water, thereby achieving the objective described above.

Still preferably, in bathing water according to the present invention, the nanobubbles are either of carbon dioxide nanobubbles or air nanobubbles.

Still preferably, bathing water according to the present invention includes the nanobubbles generated by either shearing microbubbles or shearing and pulverizing the microbubbles with high speed rotation.

Still preferably, bathing water according to the present invention includes the nanosized medical component generated by generating a micro-sized medical component and shearing the micro-sized medical component.

Still preferably, in bathing water according to the present invention, the water is bathtub water and the nanobubbles are carbon dioxide nanobubbles, and at least either of the carbon dioxide nanobubbles or the nanosized medical component are combined with the bathtub water and circulated into the bathtub.

Still preferably, in bathing water according to the present invention, the amount of the carbon dioxide nanobubbles generated is controlled, so that a concentration of carbon dioxide reaches a predetermined value based on a carbon dioxide concentration in bathtub water inside the bathtub and the carbon dioxide nanobubbles are circulated into the bathtub.

Therapeutic bathing water is provided, in which at least either of carbon dioxide nanobubbles or a nanosized medical component is combined with bathtub water, thereby achieving the objective described above.

Preferably, therapeutic bathing water according to the present invention includes at least either effect of absorbing either of the carbon dioxide nanobubbles or the nanosized medical component through the skin and taking it into a capillary vessel to increase blood flow and an insulin-like factor by the carbon dioxide nanobubbles; or circulating the nanosized medical component around a body to demonstrate a medicinal effect.

Hereinafter, the functions of the present invention having the structures described above will be described.

The inventors of the present invention has found that (1) nanobubbles remain longer in bathtub water compared with microbubbles, and therefore, it has a better thermal effect and washing effect for a human body. Although carbon dioxide is traditionally known to increase blood flow and improve blood circulation, the amount of carbon dioxide absorbed through the skin is minute.

In addition, the inventors of the present invention has found that (2) carbon dioxide nanobubbles are absorbable with various kinds of medical components through the skin since the absorbing amount of carbon dioxide nanobubbles through the skin is large. Although medical components extracted from herbal medicines are conventionally common, the amount absorbed through the skin is minute.

Further, the inventors of the present invention have found that (3) carbon dioxide nanobubbles are absorbed with various kinds of nanosized medical components through the skin, thereby utilizing it for treating various illnesses. Carbon dioxide nanobubbles are absorbed through the skin with conventionally known blood flow increasing effect and blood circulation improving effect and a nanosized medical component is absorbed through the skin with the medical component from various conventionally known medical components as well as medical components extracted from herbal medicines (a medical plant is called an herbal medicine in the field of pharmacy), so that treatment for various illnesses and maintenance of health are greatly expected.

The inventors of the present invention have come to create the present invention with the knowledge described above.

The present invention provides nanobubbles, such as nanosized carbon dioxide or nanosized air, as well as bathtub water combined with a nanosized medical component and introduce them into a bathtub. When a user bathes in this bathtub water, the nanobubbles or nanosized medical component contained in the bathtub water is absorbed into the user's skin, taken in capillary vessels and circulated around the body, thereby circulating a medical component according to various therapeutic purposes together with blood around the body. In addition, the medicinal action of the nanosized medical component can be effectively provided on the human body without being decomposed in a digestive organ, the liver or the kidneys as was done conventionally. Further, the medical effect of the medical component can be demonstrated all over the body. As a result, the effect of increasing blood flow and improving blood circulation of the carbon dioxide nanobubbles and a medicinal effect of the nanosized medical component can be utilized for a medical treatment and maintenance of health, so that a treatment and prevention effect for various illnesses and an effect on maintenance of health can be expected. Such illnesses include, for example, central neurologic disease, cardiovascular syndrome, metabolic disorder, digestive disorder, locomotory disorder, and cutaneous disorder. Typical examples of central nurologic disease include Alzheimer's disease and dementia. Cardiovascular syndrome includes chronic cardiac failure, hyperpiesia, brain infarction and cardiac infarction. Further, metabolic disorder includes gastric ulcer and deterioration in liver function. Further, locomotory disorder includes arthrorheumatism and arthritis. Further, Cutaneous disorder includes skin aging and hair loss.

(2) The nanosized medical component is generated by a medical component tub and a nano-liquid generator configured with a liquid-liquid mixture circulating pump having a micro-liquid generating section and a liquid shearing section and a needle pump. The liquid-liquid mixture circulating pump here indicates a pump that mixes and circulates two kinds of liquids.

(3) The nanobubbles of carbon dioxide or air are generated by a nanobubble generator configured with a gas-liquid mixture circulating pump having a microbubble generating section and with a gas-liquid shearing section. Alternatively, the nanobubbles are generated utilizing a microbubble generator configured with a gas-liquid mixture circulating pump having a microbubble generating section and ultra high speed rotating section that shears and pulverizes microbubbles. The gas-liquid mixture circulating pump here indicates a pump that mixes and circulates gas and liquid.

(4) The bathtub apparatus generates nanobubbles such as carbon dioxide nanobubbles and air nanobubbles with high discharge pressure and nanosized medical component containing bathtub water and introduces them into a bathtub. When a user bathes in this bathtub water, the carbon dioxide nanobubbles and nanosized medical component are absorbed into the user's skin, taken in capillary vessels and circulated around the body, so that treatment and prevention for various illnesses and maintenance of health can be expected utilizing a blood flow increasing effect of the nanobubbles and a medical action of the nanosized medical component. The bathtub apparatus combines a system for generating carbon dioxide nanobubbles from carbon dioxide generated from a liquefied carbon dioxide cylinder to be absorbed through the skin to increase blood flow and a system for generating a nanosized medical component to demonstrate a medicinal action of the medical component, allowing of obtaining a combined effect for a treatment.

For example, When a user bathes in bathtub water containing carbon dioxide nanobubbles and nanosized medical component, the carbon dioxide nanobubbles and nanosized medical component are absorbed into the user's skin, taken in capillary vessels and circulated around the body, allowing of increasing blood flow in the body by the carbon dioxide nanobubbles as well as increasing IGF-1, Insulin-like growth factor-1, which is effective for various blood-related illnesses. Information that an increase on IGF-1 (Insulin-like growth factor-1) is effective for various illnesses is being accepted in the recent medical field.

For example, Non-patent Reference 1 defines with respect to IGF-1 that “IGF-1 transfers a signal similar to insulin that adjusts blood glucose, to decrease blood sugar is important for diabetes treatment. Besides, IGF-1 is further applicable for protein degradation control, reduction of blood pressure, remedy for heart function, remedy for hyperlipidemia, increase of cognitive function, antidepressant, prevention of Alzheimer's disease, skin care, hair growth, healing of wound, anti-inflammatory effect, immune function activation effect due to activation of natural killer cells”.

As a function of IGF-1, antidepressant effect, improvement in cognitive function, remedy for Alzheimer's disease are listed for central neurologic system, for example. For cardiovascular system, cardiotonic effect, decrease in blood pressure, and controlling of arteriosclerosis are listed. For metabolism, improvement in diabetes and improvement in insulin resistance are listed. For digestive system, controlling of gastric ulcer and increase in hepatic function are listed. For blood and immune systems, promotion of production of red blood cells and improved immune activity are listed. For genitourinary system, improved reproductive function is listed. For musculoskeletal system, increase in the amount of muscle and increase in bone density are listed. Further, for skin and hair, improvement on wrinkles and loose facial skin and promotion of hair growth are listed. The information described above is based on a lecture on microbubbles by Prof. Okajima of Nagoya City University held in August, 2006, as an explanation of effects of microbubbles. However, no information was included at that time regarding “carbon dioxide nanobubbles and a nanosized medical component” generated by a nanobubble generator. Further, Non-patent Reference 1 described above does not contain any description regarding a blood flow increasing effect due to a combination of carbon dioxide nanobubbles and a nanosized medical component or a treatment and prevention for various illnesses.

According to the present invention, microbubbles at the time of their appearance have a diameter of 10 μm to several tens μm (microbubbles will turn into micro-nanobubbles due to contraction after their appearance). In addition, micro-nanobubbles have a diameter of 10 μm to several hundreds nm. Further, nanobubbles are defined to have a diameter with several hundreds nm or less. These are defined by Prof. Onari at Tokuyama Technical Junior College.

As described above, according to the present invention, nanobubbles, such as carbon dioxide nanobubbles and air nanobubbles, and a nanosized medical component, such as a nanosized herbal medicine, are generated and combined with bathing water. When a user bathes in this bathtub water, the carbon dioxide nanobubbles or nanosized medical component contained in the bathtub water is absorbed into the user's skin, taken in capillary vessels and circulated around the body, allowing of obtaining a therapeutic effect and prevention effect for various illnesses.

For example, carbon dioxide nanobubbles and air nanobubbles can increase blood flow and improve blood circulation in a human body. In addition, while carbon dioxide can increase blood flow and improve blood circulation in a human body, it can increase an insulin-like growth factor, which is effective for various illnesses. Further, when nanosized medical component is absorbed into the user's skin and is taken in capillary vessels, the medical component is effectively functioned at an action site.

When a medical component is taken internally, a portion of it is decomposed in a digestive organ, the liver or the kidneys. However, such a medical component will not be decomposed when absorbed through the skin, allowing a less amount of the medical component to demonstrate its effect. Further, since bathing is a daily activity for a patient, it can be used as a therapeutic method and a prevention method with almost no antipathy.

Microbubbles are generated and sheared with a nanobubble generator having a gas-liquid shearing section, allowing nanobubbles to be certainly generated from carbon dioxide or air. Microbubbles are also generated by a microbubble generator and are sheared and pulverized by being introduced into an ultra high-speed rotating section, allowing nanobubbles to be easily and certainly generated. Further, a micro-liquid generator can be used instead of a nano-liquid generator, and a microbubble generator can be used instead of a nanobubble generator. An economical system can be constructed with micro-liquid generator and microbubble generator since they can significantly reduce costs.

In addition, a micro-sized medical component is generated and sheared with a nano-liquid generator having a liquid shearing section, allowing the medical component to be easily and certainly nanosized.

Further, microbubbles are sheared by a nano-gas-liquid generator having a gas-liquid shearing section to generate nanobubbles and micro-liquid is sheared to generate a nanosized medical component easily and certainly.

A dissolved carbon dioxide analyzer is provided inside a bathtub and the amount of carbon dioxide introduced into a nanobubble generator is automatically controlled and adjusted using a electric-powered needle valve by a dissolved carbon dioxide controller according to a signal or signal level from the dissolved carbon dioxide analyzer, allowing a desired concentration of a dissolved carbon dioxide inside the bathtub to be set and a concentration of a dissolved carbon dioxide which a therapeutic effect and prevention effect can be obtained to be automatically set.

Further, a medical component tub can be filled with an herbal medicine, such as iris leaf, citrus, angelica root, chamomilla, cnidium rhizome, citrus unshiu peel, and ginseng, and a medical component extracted from the herbal medicine can be nanosized. Therefore, the nanosized medical component extracted from the herbal medicine can be absorbed through the skin, allowing of treating and preventing various illnesses. Further, the nanosized medical component extracted from the herbal medicine can increase blood flow, and the medical component that the herbal medicine contains can treat or prevent various illnesses. Prevention of the various illnesses means to increase the strength of the immune system to prevent such illnesses.

Further, a heater and a thermometer can be provided for the medical component tub to control the temperature (water temperature) of the medical component tub using a temperature controller, allowing the medical component to be extracted from herbal medicine in appropriate conditions so as to fill the medical component tub.

These and other advantages of the present invention will become apparent to those skilled in the art upon reading and understanding the following detailed description with reference to the accompanying figures.

### BRIEF DESCRIPTION OF THE DRAWINGS

FIG. 1 is a schematic view showing an essential structure of a bathtub apparatus according to Embodiment 1 of the present invention.

FIG. 2 is a schematic view showing an essential structure of a bathtub apparatus according to Embodiment 2 of the present invention.

FIG. 3 is a schematic view showing an essential structure of a bathtub apparatus according to Embodiment 3 of the present invention.

FIG. 4 is a schematic view showing an essential structure of a bathtub apparatus according to Embodiment 4 of the present invention.

FIG. 5 is a schematic view showing an essential structure of a bathtub apparatus according to Embodiment 5 of the present invention.

FIG. 6 is a schematic view showing an essential structure of a bathtub apparatus according to Embodiment 6 of the present invention.

FIG. 7 is a schematic view showing an essential structure of a bathtub apparatus according to Embodiment 7 of the present invention.

10 bathtub
11 hot water supply valve
20 nanobubble generator
20a nano-gas-liquid generator
20b microbubble generator
21 liquefied carbon dioxide cylinder
22 pressure-reducing valve
23, 33 needle valve
23a, 23b, 33a electric-powered needle valve
24 microbubble generating section
24a micro-gas-liquid generating section
25, 25a gas-liquid mixture circulating pump
26, 26a gas-liquid shearing section
27, 29a valve
28 carbon dioxide nanobubble discharge opening
28a air nanobubble discharge opening
28b nano-gas-liquid discharge opening
29 ultra high speed rotating section (carbon dioxide nanobubble generating section)
30 nano-liquid generator
31 medical component tub
31a medicinal plant
32 medical component tub pump
34 micro-liquid generating section
35 liquid-liquid mixture circulating pump
36 liquid shearing section
37 nanosized medical component discharge opening
38 electric-powered needle valve
41 hot water supply pipe
42 carbon dioxide pipe
43, 46 bathtub suction pipe
44, 47 bathtub water discharge pipe
45 medical component pipe
51 timer
52 dissolved carbon dioxide analyzer
53 dissolved carbon dioxide controller
61, 62 signal line
63 blower
64 electric valve
100, 100A, 100B, 100C, 100D, 100E, 100F bathtub apparatus
A water surface
B nanobubble stream
C nano-liquid stream

### DESCRIPTION OF THE PREFERRED EMBODIMENTS

Embodiments 1 to 7 of a bathtub apparatus and bathing method according to the present invention will be described in detail in reference to the attached drawings.

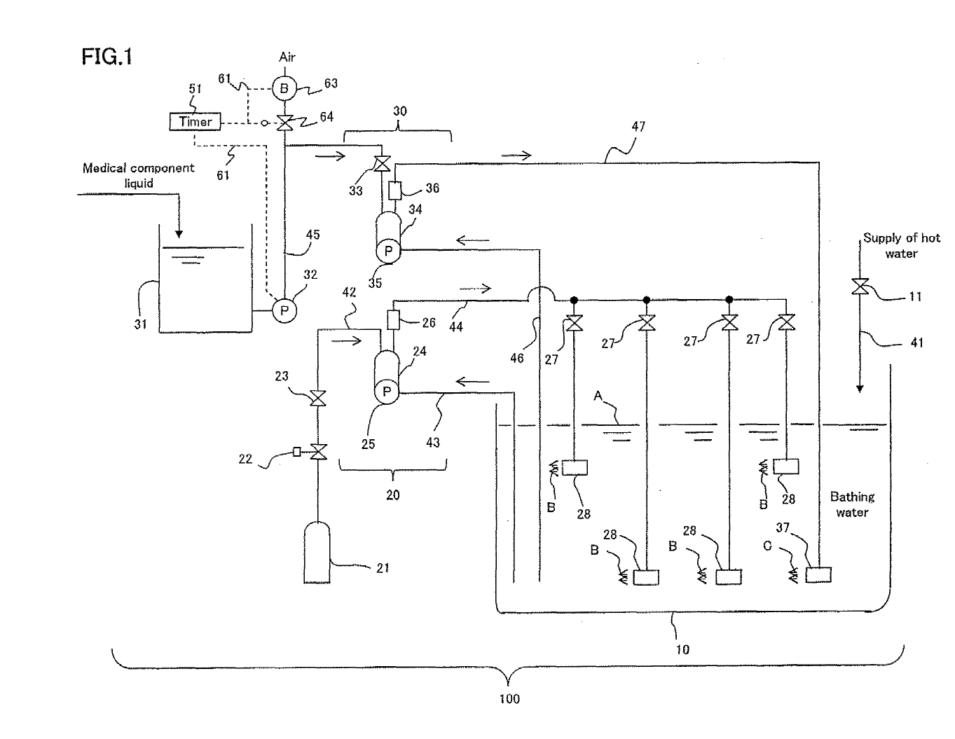

In regard to FIGS. 1-3 and FIGS. 5-7, although there is no such word as nano-liquid generator, micro-liquid generating section, liquid-liquid mixture circulating pump or liquid shearing section in the art, such words are used in the present application to clearly distinguish the generation of nano-liquid and generation of nano-gas (nanobubbles), and a nano-liquid generator 30 (micro-liquid generating section 34, liquid-liquid mixture circulating pump 35, liquid shearing section 36) and a nanobubble generator 20 (gas-liquid mixture circulating pump 25 including a microbubble generating section 24, gas-liquid shearing section 26) are described to explain the distinction expediently, in consideration of their functions. In practice, a timer 51 performs a sequence control on a medical component tub pump 32, a blower 63 for an air intake, and an electric valve 64, which are connected via control lines at respective control terminals as shown in Figures; and air (herein air taken in from the blower 63) is always needed in such a case as this for producing a nanosized medical component liquid (nano-liquid). That is, both gas and liquid are necessary, and when gas and liquid are rotated together at a high speed, difference of rotations between the gas and the liquid are created due to the difference between their specific gravities, causing them to be sheared. Therefore, the liquid-liquid mixture circulating pump 35 having a micro-liquid generating section 34, which generates micro-liquid indicated in the present application, is actually the same as a gas-liquid mixture circulating pump 35 (a gas-liquid mixture circulating pump 35 is also called a gas mixture circulating pump in the art) having a micro-liquid generating section 34, which generates microbubbles with air taken from the blower 63; and a liquid shearing section 36 is the same as a gas shearing section 36. Accordingly, a nano-liquid generator 30 is the same as a nanobubble generator 30 that is similar to a nanobubble generator 20. As mentioned before, the-liquid generator 30 and the nanobubble generator 20 are distinguished differently from each other and described as such in the present application. Further described from the point of this sequence control, a gas-liquid mixture circulating pump 35, which mixes and circulates a medical component liquid and bathing water, actually operates as a gas-liquid mixture circulating pump 35, where a medical component tub pump 32, a blower 63 and an electric valve 64 sequentially function to provide a medical component liquid and air.

Similarly, the present application will describe the nanobubble generator 20 (gas-liquid mixture circulating pump 25 including a microbubble generating section 24, gas-liquid shearing section 26) expediently considering its functions. However, in regard to the nanobubble generator 20 (gas-liquid mixture circulating pump 25 including a microbubble generating section 24, gas-liquid shearing section 26) described herein, the gas-liquid shearing section 26 is actually a gas shearing section 26, which is a component of the nanobubble generator 20, the gas-liquid mixture circulating pump 25, which includes a microbubble generating section 24, and the gas-liquid shearing section 26.

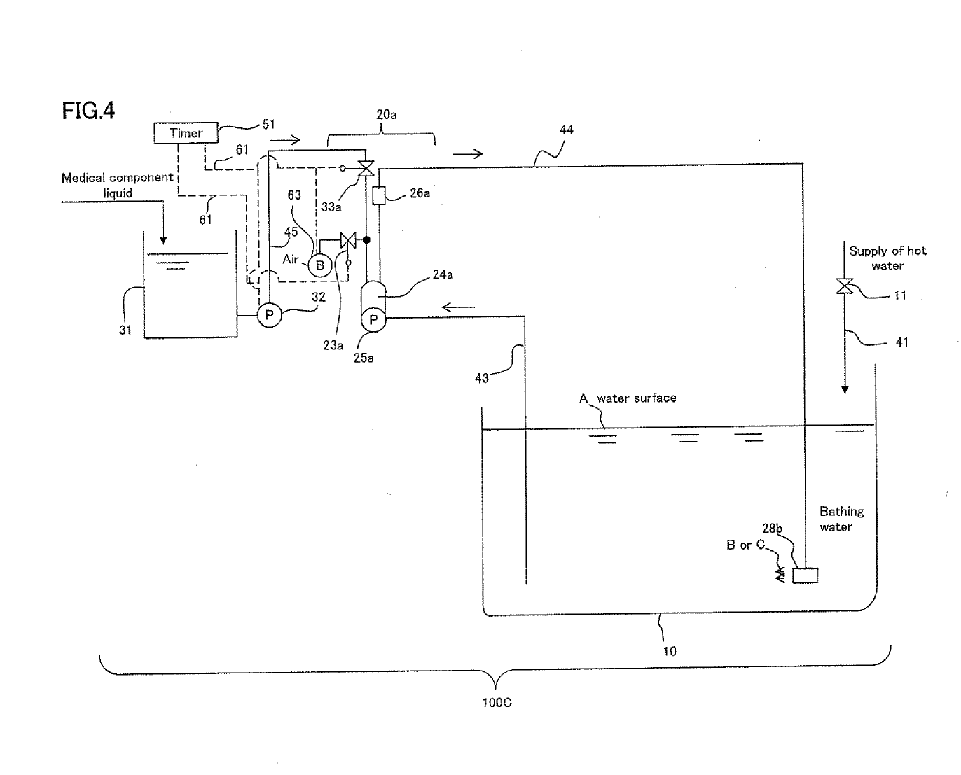

Next, in FIG. 4 (Embodiment 4), the present application will describe a nano-gas-liquid generator 20a (gas-liquid mixture circulating pump 25a having a micro gas-liquid generating section 24a, gas-liquid shearing section 26a, nano-gas/liquid discharge opening 28b) expediently, considering its function, in order to clearly distinguish the generation of nano-liquid and the generation of nano-gas (nanobubbles) by the same pump in the present application. However, the gas-liquid mixture circulating pump 25a having a micro gas-liquid generating section 24a is actually the same as a gas-liquid mixture circulating pump 25a having a microbubble generator 24a (the gas-liquid mixture circulating pump 25a is called a gas mixture circulating pump in the art), the gas-liquid shearing section is the same as a gas shearing section 26a, and a nano-gas/liquid discharge opening 28b is the same as a bubble discharge opening 28b.

According to the present application, a nanosized medical component is the same as medical component nanobubbles, and the medical component nanobubbles are bubbles which contain gas inside nanobubbles (foam) that have a liquid exterior. The medical component is in a state where it is dissolved in the liquid portion on the outside.

### Embodiment 1

FIG. 1 is a schematic view showing an essential structure of a bathtub apparatus according to Embodiment 1 of the present invention.

In FIG. 1, a bathtub apparatus 100 according to Embodiment 1 includes a bathtub 10, a nanobubble generator 20, a liquefied carbon dioxide cylinder 21, a nano-liquid generator 30, and a medical component tub 31.

The bathtub 10 can be any bathtub such as a bathtub for household use and a bathtub used in hospital, hotel, inn and hot spring spa, and it can be made of a variety of materials such as wood, stone, synthetic resin, stainless steel. In general, a bathtub for household use is made of synthetic resin and stainless steel. In addition, the bathtub 10 is provided with hot water via a hot water supply valve 11 and a hot water supply pipe 41.

The nanobubble generator 20 includes a needle valve 23 provided with gas from the liquefied carbon dioxide cylinder 21 via a pressure-reducing valve 22, a gas-liquid mixture circulating pump 25, which is connected to the needle valve 23 via a pipe 42 and has a microbubble generator 24, and a gas-liquid shearing section 26 connected to the gas-liquid mixture circulating pump 25. The gas-liquid mixture circulating pump 25 is provided with bathtub water from the bathtub 10 via the pipe 43. In addition, the gas-liquid shearing section 26 is connected to the nanobubble discharge section 28 provided inside the bathtub 10 via a pipe 44 and a valve 27. In this case, for example, four nanobubble discharge sections 28 are positioned inside the bathtub 10, and each of the nanobubble discharge sections 28 is connected to one of a plurality of the valves 27 controlled independently with each other.

The gas-liquid mixture circulating pump 25 is a pump which mixes gas and liquid and circulates them, and additionally, it is a pump capable of generating microbubbles within the pump itself. Although a pump section and a microbubble generating section have been conventionally configured separately, a special pump to which a microbubble generator 24 is attached is used herein as the gas-liquid mixture circulating pump 25. The nanobubble generator 20 according to Embodiment 1 includes a gas-liquid mixture circulating pump 25, a microbubble generator 24, gas-liquid shearing section 26, a needle valve 23 and a nanobubble discharge section 28.

As the first step for the nanobubble generator 20, microbubbles derived from carbon dioxide is generated by the gas-liquid mixture circulating pump 25 having the microbubble generator 24. In the following second step, carbon dioxide nanobubbles are generated as needed by the gas-liquid shearing section 26. At this time, the amount of carbon dioxide is accurately controlled in accordance with a degree of opening of the needle valve 23 to generate the carbon dioxide nanobubbles. A rotation rate controller (inverter) for the gas-liquid mixture circulating pump 25 may be additionally provided in the case where more accurate control is necessary. According to Embodiment 1, the amount of carbon dioxide needed for the gas-liquid mixture circulating pump 25 is preset as 0.7 litter/min., and discharge pressure from the liquefied carbon dioxide cylinder 21 is decompressed to a preset pressure level by the pressure-reducing valve 22 to increase the volume of the carbon dioxide, with the amount minutely adjusted by the needle valve 23.

The nano-liquid generator 30 includes a needle valve 33 provided with a liquid via a medical component tub pump 32 and a pipe 45 from a medical component tub 31 that retains a medical component liquid, a liquid-liquid mixture circulating pump 35 having a micro-liquid generator 34, and a liquid shearing section 36 connected to the liquid-liquid mixture circulating pump 35. The liquid-liquid mixture circulating pump 35 is provided with bathtub water from the bathtub 10 via a pipe 43. In addition, the liquid shearing section 36 is connected to a nanosized medical component discharge opening 37 placed inside the bathtub 10 via a pipe 47.

The liquid-liquid mixture circulating pump 35 is a pump which mixes and circulates two kinds of liquids (and practically including air taken in from the blower 63), the liquid being one kind of liquid (medical component liquid) and the other liquid (bathing water). In addition, the liquid-liquid mixture circulating pump 35 having a micro-liquid generator 34 is a pump capable of generating micro-liquid by the pump itself. Although a pump section and a microbubble generating section have been conventionally configured separately, a special pump to which a microbubble generator 24 is attached is used herein as the liquid-liquid mixture circulating pump 35. According to Embodiment 1, the nano-liquid generator 30 includes a liquid-liquid mixture circulating pump 35, a micro-liquid generator 34, a liquid shearing section 36, a needle valve 33, and a nanosized medical component discharge opening 37.

As the first step for the nano-liquid generator 30, micro-liquid derived from a medical component is generated by the liquid-liquid mixture circulating pump 35 having the micro-liquid generator 34. In the following second step, nano-liquid (which is practically nanobubbles since it contains air taken in from the blower 63) is generated as needed by the liquid-liquid shearing section 36. At this time, the amount of the medical component is accurately controlled in accordance with a degree of opening of the needle valve 33 to generate the nano-liquid. A rotation rate controller (inverter) for the liquid-liquid mixture circulating pump 35 may be additionally provided in the case where more accurate control is necessary. According to Embodiment 1, the amount of the medical component needed for the nano-liquid generator 30 is preset as 0.7 litter/min.

Accordingly, in Embodiment 1, a nanobubble and/or nanosized medical component generating section is configured with the gas-liquid mixture circulating pump 25 for producing cloudy water full of microbubbles by mixing and circulating bathtub water from a bathtub and gas from outside using the microbubble generating section 24, the gas-liquid shearing section 26 for shearing microbubbles provided from the gas-liquid mixture circulating pump 25 to generate nanobubbles, the liquid-liquid mixture circulating pump 35 for producing cloudy water of micro-liquid (which is practically the same as microbubbles since it contains air taken in from the blower 63) by mixing and circulating bathtub water from the bathtub and medical component from outside with the micro-liquid generator 34, and the liquid shearing section 36 for shearing micro-liquid provided from the liquid-liquid mixture circulating pump 35 to generate nano-liquid, thereby combining at least either of nanobubbles or a nanosized medical component with bath water from the bathtub 10 and circulating the bath water to the bathtub 10.

With the configuration described above, an operation of a therapeutic bathtub apparatus 100 according to Embodiment 1 will be described herein after.

First, the bathtub 10 is provided with hot water by opening the hot water supply valve 11. At this time, a water level for the bathtub 10 is adjusted in such a way that the bathtub 10 will be filled with water at the level of water surface A and the temperature of the water is adjusted to be in the range of 37 degrees Celsius and 42 degrees Celsius.

Next, a case will be described in detail where carbon dioxide nanobubbles are combined with bathtub water in the bathtub 10.

The bathtub water inside the bathtub 10 is introduced to the gas-liquid mixture circulating pump 25 via the pipe 43 for bathtub water, and in turn carbon dioxide as gas introduced from a carbon dioxide pipe 42 and bathtub water as a liquid are mixed to generate microbubbles. In regard to the carbon dioxide, liquefied carbon dioxide from a liquefied carbon dioxide cylinder 21 is decompressed by the pressure-reducing valve 22, and subsequently the amount of carbon dioxide is accurately adjusted by the needle valve 23 so that the carbon dioxide is introduced into the microbubble generating section 24.

The carbon dioxide microbubble containing bathtub water generated in the microbubble generating section 24 is then introduced into the gas-liquid shearing section 26 to generate carbon dioxide nanobubble containing bathtub water. This carbon dioxide nanobubble containing bathtub water goes through a bathtub water discharging pipe 44, and the discharging amount is adjusted by four valves 27. Subsequently, the carbon dioxide nanobubble containing bathtub water is discharged into the water inside the bathtub 10 as a nanobubble stream B from each of the four carbon dioxide nanobubble discharge openings 28.

When a user bathes in this bathtub 10, carbon dioxide nanobubbles contained in the bathtub water is easily absorbed into the user's skin, taken in capillary vessels and circulated around the body. Therefore, the effect of increasing blood flow, which carbon dioxide originally possesses, will be sufficiently demonstrated.

Generation of nanobubbles by the nanobubble generator 20 described above is performed by a first step and a second step in the following.

In the first step, microbubbles are formed by the microbubble generation section 24. In the microbubble generation section 24, pressure is controlled hydromechanically for the interior liquid, so that gas is sucked in from a negative pressure forming section. A negative pressure section is formed by employing a high speed fluid motion (bathtub water is pumped by a gas-liquid mixture pump to a microbubble generating section to form a negative pressure section inside), thereby generating microbubbles. It is said, for the sake of simplicity, that the bathtub water and carbon dioxide are effectively supplied, mixed and dissolved together and are subsequently pumped, so that cloudy water of carbon dioxide microbubble is provided.

In the second step, the microbubbles generated by the microbubble generating section 24 are introduced into the gas-liquid shearing section 26 via a pipe and are sheared employing a fluid motion, generating carbon dioxide nanobubbles that are finer than carbon dioxide microbubbles.

Next, a case will be described in detail where nanosized medical component is combined with bathtub water in the bathtub 10.

A medical component refers to a medicinal substance whose effect is granted by the pharmaceutical affairs law, and it is basically assumed that all the substances that have a medicinal action or medical effect fall under that category. In addition, a new substance that is absorbed through the skin and shows effective medicinal action may be developed in the future, and it is needless to say that such a substance will also fall under the same category. Many of the existing medical components fall under the category of pharmaceuticals granted by the pharmaceutical affairs law. There are a great number of medical components that fall under the category of pharmaceuticals, and they are listed on Japanese Pharmacopoeia, for example. Forms of the medical components can include but not limited to liquids such as a liquid medicine and powder such as powdered drug. However, a component dissolvable into water is suitable since it is combined with bathtub water and is easy to be nanosized.

Bathtub water in the bathtub 10 is introduced from a pipe 46 for bathtub water into the liquid-liquid mixture circulating pump 35, and a medical component as liquid introduced from a pipe 45 for a medical component and bathtub water as liquid are mixed (and air from the blower 63 is also mixed substantially although this is described as a mixture of liquids), generating medical component micro-liquid (or it could be considered as medical component microbubbles since air is added in practice). A medical component liquid, to which the medical component described above is added, is retained in the medical component tub 31. The medical component liquid out of the medical component tub 31 is pumped by the medical component tub pump 32, where the amount of the medical component liquid is accurately adjusted by the needle valve 33, and is subsequently introduced into the micro-liquid generation section 34.

Medical component micro-liquid containing bathtub water generated in the micro-liquid generation section 34 is, in turn, introduced into the liquid shearing section 36 to generate medical component nano-liquid containing bathtub water. This medical component nano-liquid containing bathtub water go through a bathtub water discharging pipe 47 and discharged from a nanosized medical component discharge opening 37 as a nano-liquid stream C into the bathtub 10.

When a user bathes in this bathtub 10, the nanosized medical component contained in the bathtub water is easily absorbed into the user's skin, taken in capillary vessels and circulated around the body. Therefore, the medicinal action that the medical component originally possesses will be sufficiently demonstrated.

Generation of nanosized medical component by the nano-liquid generator 30 described above is performed by a first step and a second step in the following.

In the first step, the medical component micro-liquid is formed by the micro-liquid generation section 34. In the micro-liquid generation section 34, pressure is controlled hydromechanically for the interior liquid, so that a medical component liquid is sucked in from a negative pressure forming section. A negative pressure section is formed employing a high speed fluid motion (bathtub water is pumped to the micro-liquid generating section by the liquid-liquid mixture pump, so that the negative pressure section is formed), thereby generating medical component micro-liquid. It is said, for the sake of simplicity, that the bathtub water and the medical component liquid are effectively supplied, mixed and dissolved together and are subsequently pumped, so that cloudy water of medical component micro-liquid is provided.

In the second step, the medical component micro-liquid generated by the micro-liquid generating section 34 is introduced into the liquid shearing section 36 via a pipe and is sheared employing a fluid motion, generating medical component nano-liquid that is finer than medical component micro-liquid.

Three kinds of bubbles will be explained here. Ordinary bubbles (gas bubble) ascend in the water and consequently burst and disappear at the surface. Microbubbles, which are minute gas bubbles of 10 μm to several tens μm, are contracted in the water and they eventually disappear (completely dissolved). Further, nanobubbles are bubbles that are even smaller than microbubbles, the diameter of which is less than or equal to 1 μm, that is 100 nm to 200 nm (10 μm to several hundred nm), and they are able to exist in the water for any length of time. Additionally, micro-nanobubbles are bubbles of the mixture of microbubbles and nanobubbles.

As described above, according to the bathtub apparatus 100 of Embodiment 1, when a user bathes in the bathtub 10 having bathtub water combined with the carbon dioxide nanobubbles and the nanosized medical component, the carbon dioxide nanobubbles and the nanosized medical component are absorbed through the skin and are taken in capillary vessel. Consequently, the medical component functions effectively, helping to promote the maintenance of health as well as performing a certain degree of therapy for the human body.

Conventionally, neither carbon dioxide nor a medical component can be absorbed through the skin; however generating nanosized carbon dioxide and a medical component allows them to be easily absorbed through the skin. Such carbon dioxide and a medical component can easily reach action sites of the human body through a capillary vessel without being decomposed in a digestive organ, the liver or the kidneys, and have effects to work well against various illnesses.

In addition, carbon dioxide nanobubbles can increase blood flow and contribute a insulin-like growth factor to recover vital functions such as immune function. Concurrently, in a case where a medicinal substance is taken, the carbon dioxide nanobubbles have an effect to increase the effectiveness of the medicinal substance, and therefore, it can be expected that a lower amount of the medicinal substance can be sufficient in providing an equal effectiveness.

Further, the bathtub apparatus 100 is not only a therapeutic bathtub apparatus where therapeutic effects can be expected, but it also washes the human body. Therefore, the bathtub apparatus 100 can be used for face and hair for cosmetic purposes, lowering the amount of shampoo or body soap used.

Actual effects of Embodiment 1 are described in the following. The following comparison experiment was conducted using the bathtub apparatus 100 shown in FIG. 1 as an apparatus used for the experiment. The bathtub apparatus 100 was configured to have a bathtub 10 with a volume of 2 m³, a motor for a liquid-liquid mixture circulating pump 35 with a power of 3.7 kw, and a motor for a gas-liquid mixture circulating pump 25 with a power of 3.7 kw. For a patient who suffers from diabetes for ten years or longer and practices medicinal treatment, dietetic therapy and kinesitherapy strictly, a comparison on his fasting blood sugar level and his sugar level after meal was made before and after a bathing therapy with the bathtub apparatus 100. Although the resulting level differed from day to day, his blood sugar level was lowered by 30% to 60% on average. Not only the blood sugar level but also various other clinical laboratory examinations with effective results were obtained from experimentation data from the Japanese Red Cross Hospital (Nisseki Hospital) and other medical offices belonging to enterprises. For example, a significant improvement was recognized in the secretion of insulin for a certain period of time in a glucose tolerance test.

### Embodiment 2

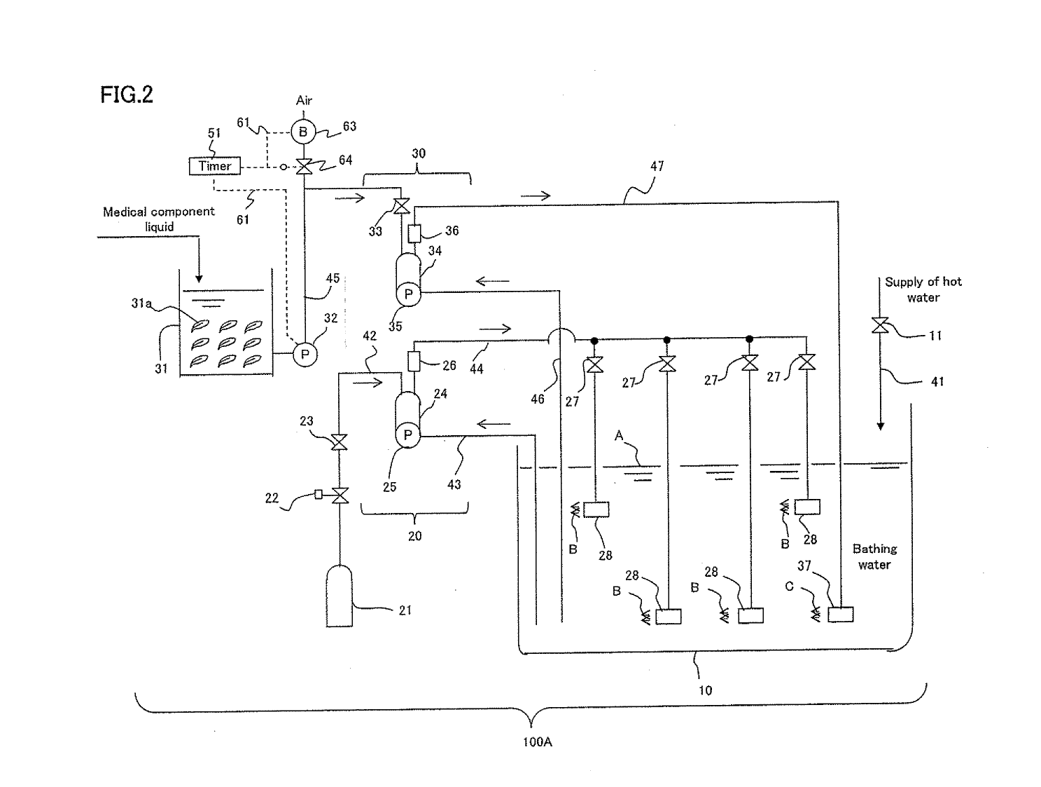

FIG. 2 is a schematic view showing an essential structure of a bathtub apparatus according to Embodiment 2 of the present invention. Constructional elements of the bathtub apparatus in FIG. 2 having the same function or effect as those of the bathtub apparatus 100 shown in FIG. 1 are designated with the same reference numerals respectively, and descriptions for such elements will be omitted. Instead, only constructional elements that differ from those in Embodiment 1 described above will be described in detail herein after.

With respect to the bathtub apparatus 100A according to Embodiment 2 in FIG. 2, herbal medicine described as a medicinal plant 31a, which is not used in the bathtub apparatus 100 according to Embodiment 1 shown in FIG. 1, is used as a filler inside a medical component tub 31.

For example, one of iris leaf, citrus, angelica root, chamomilla, cnidium rhizome, citrus unshiu peel, ginseng, or two or more of the combination thereof is selected as a medical plant 31a and filled in the medical component tub. Such a medicinal plant includes a medicinal plant treated as both pharmaceuticals and a folk medicine. For example, root of Achyranthes Japonica and seed of Plantago Asiatica are selected for diabetes treatment. Further, coptis rhizome, buplever, and Asiatic ginseng are selected for arteriosclerosis treatment.

A medical component is extracted from the medicinal plant 31a to generate a medical component liquid in the medical component tub 31, and the medical component liquid is transferred to a nano-liquid generator 20 by a medical component pump 32. As a result, the medical component from the medicinal plant 31a is nanosized to produce a nanosized medical component.

As described above with respect to the bathtub apparatus 100A according to Embodiment 2 as a therapeutic bathtub apparatus, the medical component tub 31 is filled with medicinal plant (herbal medicine) 31a to extract a medical component in accordance with a purpose, the medical component combined with bathtub water. In bathing, a nanosized medical component together with carbon dioxide nanobubbles is largely absorbed through the skin, taken in capillary vessels and circulated around the body. Therefore, a medicinal effect of herbal medicine can be efficiently used for therapy.

A therapeutic method of putting in a medicinal plant in a bathtub and taking a bath with a medical component extracted therein has conventionally existed. However, a method of absorbing a medical component through the skin together with carbon dioxide nanobubbles has not previously existed. In addition, a medical component can be extracted from the medicinal plant 31a in the medical component tub 31 taking a long period of time. Further, a heater and a thermometer can be provided for the medical component tub 31, and a temperature controller can be connected to the heater to control the heater in response to the temperature of the medical component tub 31 measured by the thermometer, allowing the temperature of the medical component tub 31 to be controlled and adjusted. By adjusting the temperature of the medical component tub 31 as described, a medical component can be extracted in an appropriate extract condition from the medicinal plant 31a that fills the medical component tub 31.

### Embodiment 3

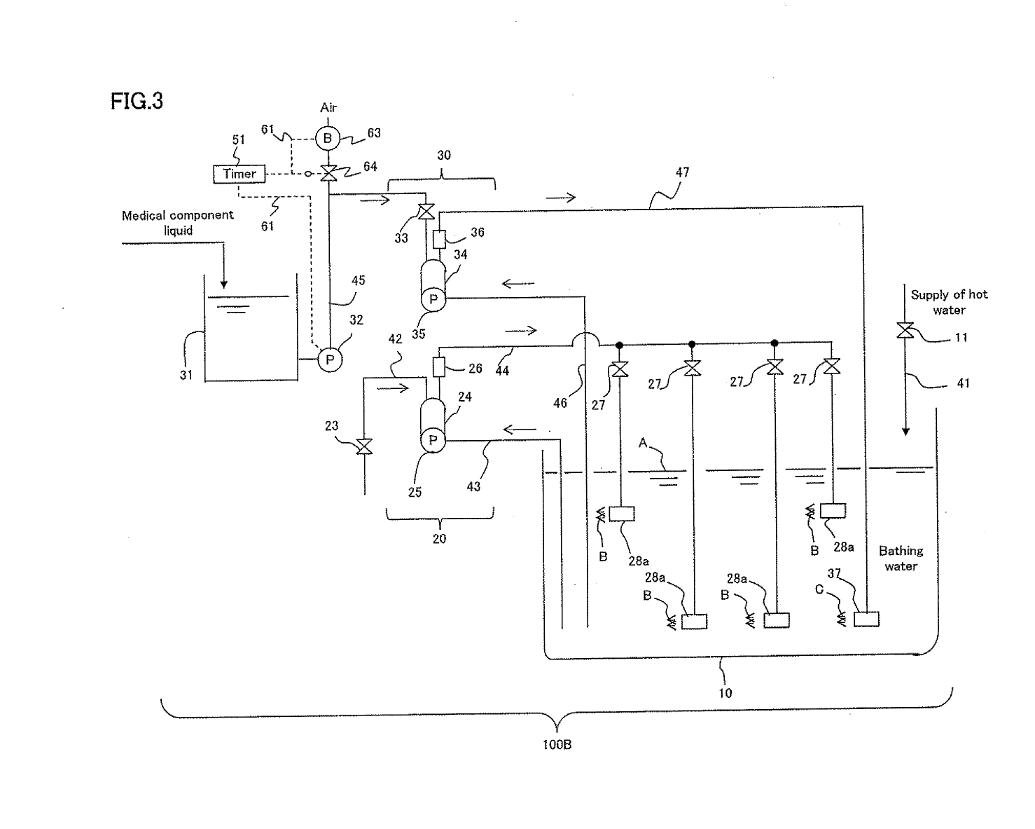

FIG. 3 is a schematic view showing an essential structure of a bathtub apparatus 100 according to Embodiment 3 of the present invention. Constructional elements of the bathtub apparatus in FIG. 3 having the same function or effect as those of the bathtub apparatus 100 shown in FIG. 1 are designated with the same reference numerals respectively, and descriptions for such elements will be omitted. Instead, only constructional elements that differ from those in Embodiment 1 described above will be described in detail herein after.

In FIG. 3, compared with the bathtub apparatus 100 according to Embodiment 1 shown in FIG. 1, a liquefied carbon dioxide cylinder 21 for providing carbon dioxide and a pressure-reducing valve 22 are removed from a bathtub apparatus 100B according to Embodiment 3, and an air nanobubble discharge opening 28a is provided instead of a carbon dioxide nanobubble discharge opening 28.

Because neither a pressure-reducing valve 22 nor a liquefied carbon dioxide cylinder 21 is provided in the bathtub apparatus 100B, air is provided for a gas-liquid mixture circulating pump 25 having a microbubble generator 24 via a needle valve 23 to generate air nanobubbles, the air nanobubbles discharged from an air nanobubble discharge opening 28a as an air nanobubble stream B. The air nanobubbles may not be as effective for increasing blood flow as carbon dioxide nanobubbles, however it has a certain degree of an increase effect on blood flow.

As described above with respect to the bathtub apparatus 100B according to Embodiment 3 as a therapeutic bathtub apparatus, nanobubbles increase blood flow and the medicinal effect by the nanosized medical component is demonstrated, and therefore a combined effect of the air nanobubbles and nanosized medical compound can be obtained.

### Embodiment 4

FIG. 4 is a schematic view showing an essential structure of a bathtub apparatus according to Embodiment 4 of the present invention. Constructional elements of the bathtub apparatus in FIG. 4 having the same function or effect as those of the bathtub apparatus 100 shown in FIG. 1 are designated with the same reference numerals respectively, and descriptions for such elements will be omitted. Instead, only constructional elements that differ from those in Embodiment 1 described above will be described in detail herein after.

In FIG. 4, compared with the bathtub apparatus 100 according to Embodiment 1 shown in FIG. 1, a liquefied carbon dioxide cylinder 21, a pressure-reducing valve 22, a nanobubble generator 20, and four carbon dioxide nanobubble discharge openings 28 are removed from a bathtub apparatus 100C according to Embodiment 4, and a nano-gas-liquid generator 20a, which generate either or both of a nanosized gas (air nanobubble) 65- and a nanosized liquid (nanosized medical component), and a nano-gas/liquid discharge opening 28b connected to the nano-gas-liquid generator 20a are provided instead of a nano-liquid generator 30.

More specifically, a gas-liquid mixture circulating pump 25a having a micro-gas-liquid generation section 24a as a microbubble/micro-liquid generation section is provided instead of a liquid-liquid mixture circulating pump 35 having a micro-liquid generating section 34; an electric-powered needle valve 23a and an electric-powered needle valve 33a are provided instead of a needle valve 33; and a timer 51 is added. The two electric-powered needle valves 23a and 33a are configured to receive a sequence control by the timer 51 via a signal line 61 and to act reciprocally in cooperation with each other.

Air is provided for the gas-liquid mixture circulating pump 25a having a micro-gas-liquid generation section 24a via the electric-powered needle valve 23a to generate air nanobubbles, the air nanobubbles discharged from the nano-gas-liquid discharge opening 28b as a nanobubble stream B. In addition, a medical component from the medical component tub 31 is provided for the gas-liquid mixture circulating pump 25a having a micro-gas-liquid generation section 24a via a medical component tub pomp 32 and the electric-powered needle valve 33a to generate a nanosized medical component, the nanosized medical component discharged from the nano-gas-liquid discharge opening 28b as a nano-liquid stream C.

Duration for generation of the nanosized medical component and for generation of the air nanobubbles may be optionally determined by the timer 51, and an operational method for the timer 51 may be set depending on a kind of target illness.

Accordingly, in Embodiment 4, a nanobubble and/or nanosized medical component generating section is configured with the gas-liquid mixture circulating pump 25a as a gas-liquid/liquid-liquid mixture circulating pump for producing cloudy water full of microbubbles or micro-liquid by mixing and shearing bathtub water from the bathtub 10 and air or a medical component liquid from outside using the microbubble generating section 24; and the gas-liquid shearing section 26a for shearing microbubbles or a micro-liquid provided from the gas-liquid mixture circulating pump 25a to generate nanobubbles or nano-liquid, thereby combining at least either of nanobubbles or a nanosized medical component with bath water from the bathtub 10 and circulating the bath water to the bathtub 10.

As described above with respect to the bathtub apparatus 100C according to Embodiment 4 as a therapeutic bathtub apparatus, it is not necessary to provide two different generators, thereby being nano-gas generator and nano-liquid generator, because both a nanosized medical component and air nanobubbles are generated by a single nano-gas-liquid generator 20a and they are combined with bathtub water in the bathtub 10, thereby simplifying the apparatus and lowering initial cost.

It is needless to say that, in Embodiment 4, a gas cylinder can be attached to an air intake to generate carbon dioxide nanobubbles and a nanosized medical component using the nano-gas-liquid generator 20a.

### Embodiment 5

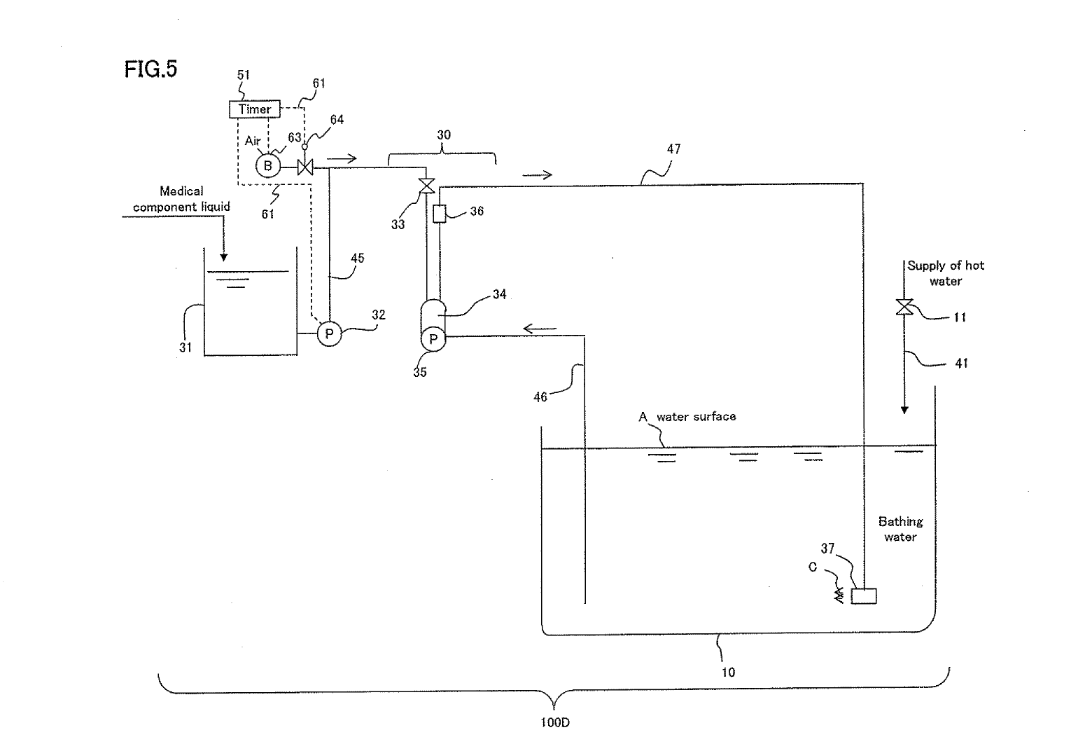

FIG. 5 is a schematic view showing an essential structure of a bathtub apparatus according to Embodiment of the present invention. Constructional elements of the bathtub apparatus in FIG. 5 having the same function or effect as those of the bathtub apparatus 100 shown in FIG. 1 are designated with the same reference numerals respectively, and descriptions for such elements will be omitted. Instead, only constructional elements that differ from those in Embodiment 1 described above will be described in detail herein after.

In FIG. 5, compared with the bathtub apparatus 100 according to Embodiment 1 shown in FIG. 1, a liquefied carbon dioxide cylinder 21, a pressure-reducing valve 22, a nanobubble generator 20, and four carbon dioxide nanobubble discharge openings 28 are removed from a bathtub apparatus loon according to Embodiment 5, and a nanosized medical component is combined with bathtub water from a bathtub 10 by a liquid-liquid mixture circulating pump 35 having a micro-liquid generating section 34 and a liquid shearing section 36 to circulate the bathtub water to the bathtub 10.

In this Embodiment, carbon dioxide nanobubbles are not generated, but instead a nanosized medical component only is generated by a nano-liquid generator 30 since none of a liquefied carbon dioxide cylinder 21, a pressure-reducing valve 22 or a nanobubble generator 20 is provided.

Accordingly, in Embodiment 5, a nanobubble and/or nanosized medical component generating section is configured with the liquid-liquid mixture circulating pump 35 for producing cloudy water full of microbubbles by mixing and shearing bathtub water from the bathtub 10 and a medical component liquid from outside using the micro-liquid generating section 34; and the liquid shearing section 36 for shearing a micro-liquid provided from the liquid-liquid mixture circulating pump 35 to generate a nano-liquid, thereby combining at least either of nanobubbles or a nanosized medical component with bath water from the bathtub 10 and circulating the bath water to the bathtub 10.

As described above with respect to the bathtub apparatus 100D according to Embodiment 5 as a therapeutic bathtub apparatus, though carbon dioxide nanobubbles are not generated, a medicinal effect due to a nanosized medical component is demonstrated, and a therapeutic effect and an effect for maintenance of health can be expected.

For using only microbubbles instead of using only a medical component liquid, although not described in Embodiment 5, only a gas-liquid mixture circulating pump 25 for producing a cloudy water full of microbubbles by mixing and shearing bathtub water from the bathtub 10 and gas from outside using a microbubble generating section 24, and a gas-liquid shearing section 26 for shearing microbubbles provided from the gas-liquid mixture circulating pump 25 to generate nanobubbles can be configured.

### Embodiment 6

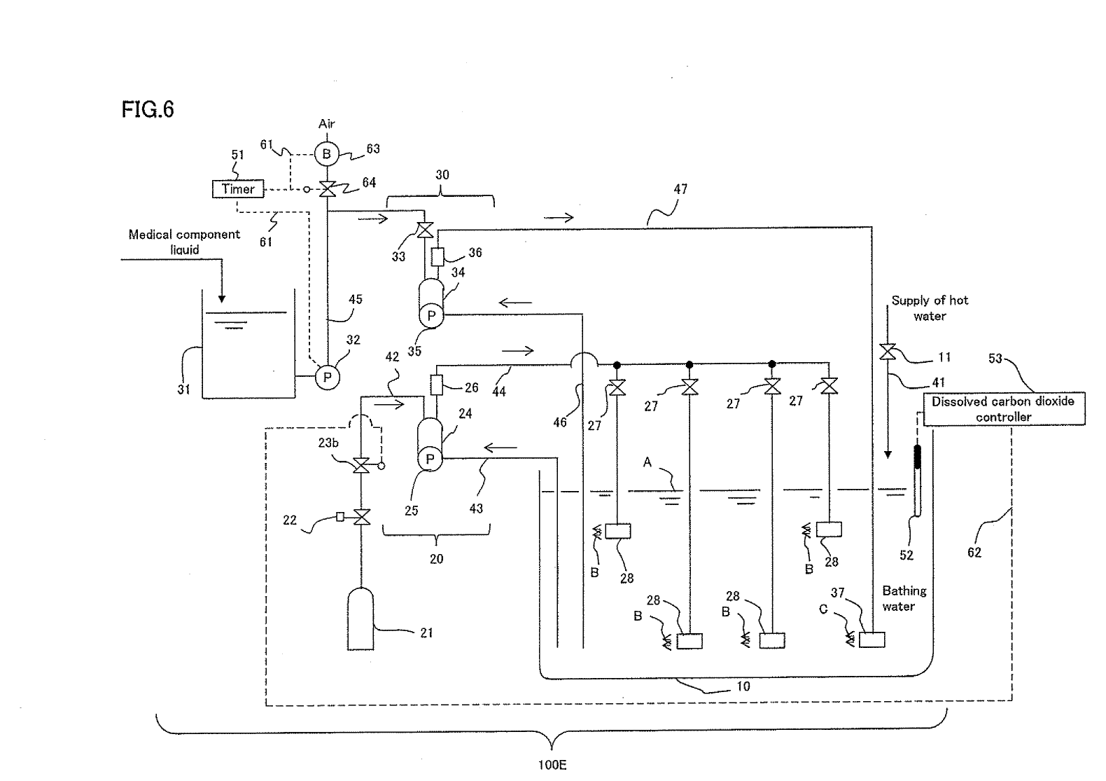

FIG. 6 is a schematic view showing an essential structure of a bathtub apparatus according to Embodiment 6 of the present invention. Constructional elements of the bathtub apparatus in FIG. 6 having the same function or effect as those of the bathtub apparatus 100 shown in FIG. 1 are designated with the same reference numerals respectively, and descriptions for such elements will be omitted. Instead, only constructional elements that differ from those in Embodiment 1 described above will be described in detail herein after.

In FIG. 6, compared with the bathtub apparatus 100 according to Embodiment 1 shown in FIG. 1, a bathtub apparatus 100E is configured with a dissolved carbon dioxide analyzer 52 positioned inside a bathtub 10 for measuring the amount of dissolved carbon dioxide, and a dissolved carbon dioxide controller 53 positioned outside the bathtub 10, both of which are connected with wiring. The bathtub apparatus 100E is configured in such a manner that the dissolved carbon dioxide controller 53 is electrically connected with an electric-powered needle valve 23b via a signal line 62, and a degree of opening of the electric-powered needle valve 23b is controlled by a controlling signal from the dissolved carbon dioxide controller 53, so that a desired concentration of a dissolved carbon dioxide in the bathtub water in the bathtub 10 can be obtained.

The dissolved carbon dioxide controller 53 electrically controls the electric-powered needle valve 23b according to the concentration of dissolved carbon dioxide in the bathtub 10 that is measured by the dissolved carbon dioxide analyzer 52, the electric-powered needle valve 23 controlling and adjusting the amount of carbon dioxide introduced into a nanobubble generator 20.

For example, the electric-powered needle valve 23b is opened if a desired concentration of a dissolved carbon dioxide is not obtained, and carbon dioxide from a liquefied carbon dioxide cylinder 21 is introduced into the nanobubble generator 20 to generate carbon dioxide nanobubbles to the maximum. When a desired concentration for the dissolved carbon dioxide is obtained, the electric-powered needle valve 23b is closed because carbon dioxide nanobubbles are no longer needed to be generated.

As described above with respect to the bathtub apparatus 100E according to Embodiment 6 as a therapeutic bathtub apparatus, the concentration of the dissolved carbon dioxide in the bathtub 10 can be maintained at an appropriate level depending on a purpose. For example, dissolved carbon dioxide is said to have a therapeutic effect when its concentration is about 1000 ppm or above according to past results in Europe, especially a number of cases in Germany, and therefore a concentration of dissolved carbon dioxide can be controlled in such a manner to be about 1000 ppm or above. Further, such a concentration of dissolved carbon dioxide can be automatically controlled by the dissolved carbon dioxide analyzer 52, the dissolved carbon dioxide controller 53 and the electric-powered needle valve 23b in order to maximize a therapeutic effect of carbon dioxide nanobubbles.

### Embodiment 7

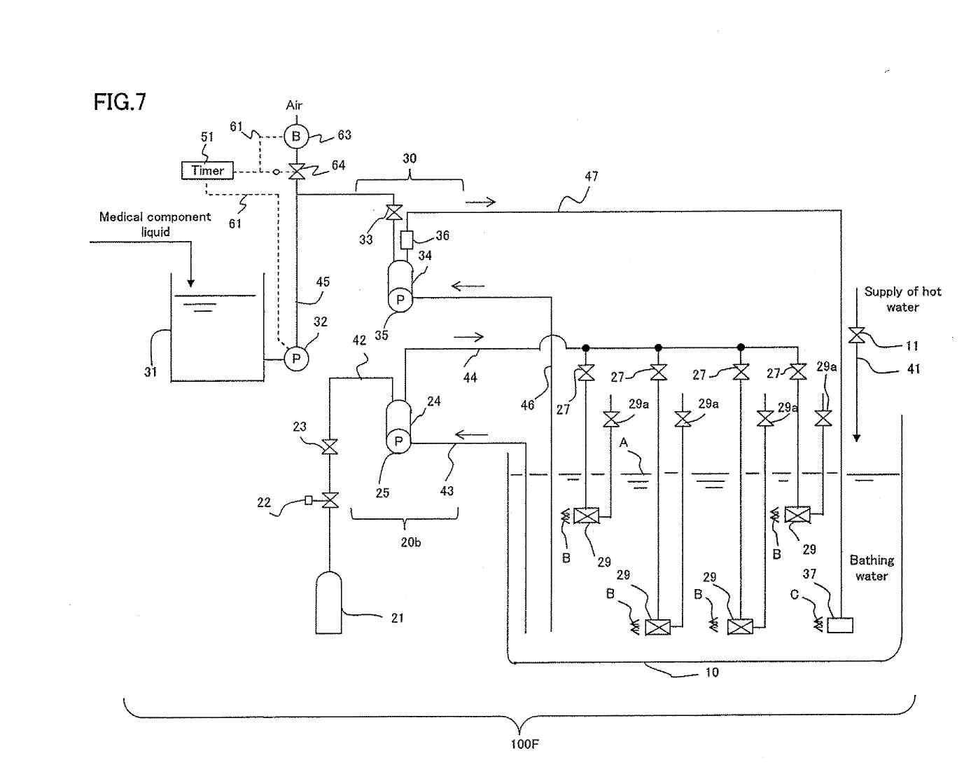

FIG. 7 is a schematic view showing an essential structure of a bathtub apparatus according to Embodiment 7 of the present invention. Constructional elements of the bathtub apparatus in FIG. 7 having the same function or effect as those of the bathtub apparatus 100 shown in FIG. 1 are designated with the same reference numerals respectively, and descriptions for such elements will be omitted. Instead, only constructional elements that differ from those in Embodiment 1 described above will be described in detail herein after.

In FIG. 7, compared with the bathtub apparatus 100 according to Embodiment 1 shown in FIG. 1, in the bathtub apparatus 100F according to Embodiment 7, a gas-liquid shearing section 26 of a nanobubble generator 20 is removed, forming a microbubble generator 20b; four ultra high-speed rotating sections 29 (carbon dioxide nanobubble generating section) are provided instead of four carbon dioxide nanobubble discharge openings 28 positioned at respective ends of a bathtub water discharging pipe 44; valves 29a are provided for the respective ultra high-speed rotating sections 29; and further, valves 27 are provided for the respective ultra high-speed rotating sections 29 on the branch side from the bathtub water discharging pipe 44.

While carbon dioxide nanobubbles are generated in the bathtub apparatus 100 according to Embodiment 1 shown in FIG. 1 at an exit point of the gas-liquid shearing section 26, carbon dioxide microbubbles are generated and outputted at the bathtub apparatus 100E according to Embodiment 7 shown in FIG. 7 by the microbubble generator 20b since a gas-liquid shearing section 26 is not provided, and the microbubbles are subsequently introduced into the ultra high-speed rotating sections 29 (carbon dioxide nanobubble generating section) to generate carbon dioxide nanobubbles.

With respect to the ultra high-speed rotating section 29 (carbon dioxide nanobubbles generating section), a rotating cavity section is formed at the center of the ultra high-speed rotating section 29, and carbon dioxide nanobubbles are generated by shear and pulverization of difference between rotating speeds at the front and back of the apparatus (the front and back of the discharge opening). Strictly speaking, air-carbon dioxide nanobubbles are generated because air is also taken in from a valve 29a at the ultra high-speed rotating section 29 (carbon dioxide nanobubbles generating section). That is, air-carbon dioxide nanobubbles are generated at the ultra high-speed rotating section 29 (carbon dioxide nanobubbles generating section) by rotating gas as nanobubbles, air and bathtub water at ultra high speed. The amount of air is adjusted by the valve 29a.

Accordingly, in Embodiment 7, a nanobubble and/or nanosized medical component generating section is configured with the gas-liquid mixture circulating pump 25 for producing cloudy water full of microbubbles by mixing and shearing bathtub water from the bathtub 10 and gas from outside using the microbubble generating section 24; the ultra high-speed rotating section 29 as a first high-speed rotating section 29, which is provided inside the bathtub 10, for shearing and pulverizing microbubbles provided from the gas-liquid mixture circulating pump 25 using difference between rotating speeds at the front and back of the discharge opening; the liquid-liquid mixture circulating pump 35 for producing cloudy water of micro-liquid by mixing and shearing bathtub water from the bathtub 10 and a medical component from outside; and the liquid shearing section 36 (which is actually the gas shearing section 36; nanobubbles and nano-liquid generated are distinguished in this context, however the same gas shearing section is practically used for both nanobubble and nano-liquid) for shearing a micro-liquid provided from the liquid-liquid mixture circulating pump 35 (and practically including air taken in from the blower 63 and microbubbles are formed from the air) to generate a nano-liquid (and practically nanobubbles are formed by shearing the microbubbles), thereby combining at least either of nanobubbles or a nanosized medical component with bath water from the bathtub 10 and circulating the bath water to the bathtub 10.

As described above with respect to the bathtub apparatus 100F according to Embodiment 7 as a therapeutic bathtub apparatus, microbubbles from the microbubble generator 20b are introduced into the ultra high-speed rotating section, allowing of certainly generating nanobubbles.

Although not described in Embodiment 7, a micro-liquid generator or a micro-gas-liquid generator can be used instead of a nano-liquid generator or a nano-gas-liquid generator. An ultra high speed rotating section 29 can be used instead of a discharge opening. In addition, a micro-sized medical component can be nanosized by the ultra high speed rotating section 29.

As a case where a ultra high speed rotating section is used on the opposite side from that of the bathtub apparatus 100F according to Embodiment 7 with respect to the high speed rotating section to be used either on the microbubble side or the micro-liquid side, there are provided a gas-liquid mixture circulating pump 25 for producing cloudy water full of microbubbles by mixing and shearing bathtub water from the bathtub 10 and gas from outside using a microbubble generating section 24; a gas-liquid shearing section 26 for shearing microbubbles provided from the gas-liquid mixture circulating pump 25 to generate nanobubbles, a liquid-liquid mixture circulating pump 35 for producing cloudy water of micro-liquid by mixing and circulating bathtub water from the bathtub and medical component from outside with the micro-liquid generator 34, and a second high speed rotating section (not shown), which is provided inside the bathtub 10, for shearing and pulverizing the micro-liquid provided from the liquid-liquid mixture circulating pump 35 by difference between rotating speeds at the front and back of the discharge opening to generate a nano-liquid.

As a different case where a high speed rotating section is provided, there can be provided only a gas-liquid mixture circulating pump 25 for producing cloudy water full of microbubbles by mixing and shearing bathtub water from the bathtub 10 and gas from outside using the microbubble generating section 24, and a high speed rotating section 29, which is provided inside the bathtub 10, for shearing and pulverizing microbubbles provided from the gas-liquid mixture circulating pump 25 using difference between rotating speeds at the front and back of the discharge opening to generate nanobubbles.

Further, there can be provided only a liquid-liquid mixture circulating pump 35 for producing cloudy water of micro-liquid by mixing and circulating bathtub water from the bathtub and medical component from outside with the micro-liquid generator 34, and a high speed rotating section (not shown), which is provided inside the bathtub 10, for shearing and pulverizing the micro-liquid provided from the liquid-liquid mixture circulating pump 35 by difference between rotating speeds at the front and back of the discharge opening to generate a nano-liquid.

Further, there can be provided a gas-liquid/liquid-liquid mixture circulating pump 25a for producing cloudy water full of microbubbles or micro-liquid by mixing and shearing bathtub water from the bathtub 10 and gas or a medical component liquid from outside using the microbubble generating section 24a, and a high speed rotating section (now shown), which is provided inside the bathtub 10, for shearing and pulverizing microbubbles or a micro-liquid provided from the gas-liquid/liquid-liquid mixture circulating pump 25a using difference between rotating speeds at the front and back of the discharge opening that employs centrifugal separation effect to generate nanobubbles or nano-liquid.

Further, there can be provided a gas-liquid mixture circulating pump 25 for producing cloudy water full of microbubbles by mixing and shearing bathtub water from the bathtub 10 and gas from outside using the microbubble generating section 24, a high speed rotating section 29, which is provided inside the bathtub 10, for shearing and pulverizing microbubbles provided from the gas-liquid mixture circulating pump 25 using difference between rotating speeds at the front and back of the discharge opening that employs a centrifugal separation effect to generate nanobubbles, a liquid-liquid mixture circulating pump 35 for producing cloudy water of micro-liquid by mixing and circulating bathtub water from the bathtub and medical component from outside with the micro-liquid generator 34, and a second high-speed rotating section (not shown), which is provided inside the bathtub 10, for shearing and pulverizing the micro-liquid provided from the liquid-liquid mixture circulating pump 35 by difference between rotating speeds at the front and back of the discharge opening to generate a nano-liquid.

According to Embodiments 1-7 described above, bathtub water, which contains nanobubbles of nanosized carbon dioxide or air and a nanosized medical component is generated to be introduced into the bathtub, and when a user bathes in this bathtub water, the carbon dioxide nanobubbles or nanosized medical component contained in the bathtub water is absorbed into the user's skin, taken in capillary vessels and circulated around the body. Nanobubbles are generated using, for example, a nanobubble generator 20 configured with a gas-liquid mixture circulating pump 25 having a microbubble generator 24 and a gas-liquid shearing section 26. Additionally/alternatively, a nanosized medical component is generated using, for example, a nano-liquid generator 30 configured with a medical component tub 31, a liquid-liquid mixture circulating pump 35 having a micro-liquid generating section 34, a liquid shearing section 36, and a needle valve 33. As a result, a bathtub apparatus, which a therapeutic effect for various illnesses and an effect for maintenance of health is expected, can be provided due to carbon dioxide and a medical component that are barely absorbed through the skin in the conventional art.

Although not described in Embodiments 1-7 described above, a nanobubble and/or nanosized medical component generating section to combine at least either of nanobubbles or a nanosized medical component with bath water from a bathtub and to circulate the bath water to the bathtub is provided, so that carbon dioxide or a medical component, which is barely absorbed through the skin conventionally, is generated as carbon dioxide nanobubbles or a nanosized medical component by a nanobubble generator or a nano-liquid generator and is combined with bathtub water. When a user bathes in this bathtub water, the carbon dioxide nanobubbles or nanosized medical component contained in the bathtub water is absorbed into the user's skin, taken in capillary vessels and circulated around the body with blood, thereby achieving the objective of the present invention, obtaining a therapeutic effect for various illnesses and an effect for maintenance of health.

As described above, the present invention is exemplified by the use of its preferred Embodiments 1 to 7. However, the present invention should not be interpreted solely based on Embodiments 1 to 7 described above. It is understood that the scope of the present invention should be interpreted solely based on the claims. It is also understood that those skilled in the art can implement equivalent scope of technology, based on the description of the present invention and common knowledge from the description of the detailed preferred Embodiments 1 to 7 of the present invention. Furthermore, it is understood that any patent, any patent application and any references cited in the present specification should be incorporated by reference in the present specification in the same manner as the contents are specifically described therein.

### INDUSTRIAL APPLICABILITY

The present invention relates to a bathtub apparatus for treating various illnesses and maintaining a health through bathing and a bathing method using the bathtub apparatus, in particular to a bathing apparatus to effectively generate carbon dioxide nanobubbles and air nanobubbles as gas as well as a nanosized medical component as liquid and to combine the nanobubbles or nanosized medical component with a bathtub water, in which the nanobubbles or medical component contained in the bathtub water is easily absorbed into the user's skin when a user bathes with this bathtub water, and thereby taken in capillary vessels and circulated around the body, thereby obtaining a therapeutic effect for various illnesses and maintaining health; it also includes a therapeutic bathtub apparatus using the bathtub apparatus, bathing water used therein, and therapeutic bathing water using the bathing water. In such a field, nanobubbles, such as carbon dioxide nanobubbles and air nanobubbles, and a nanosized medical component, such as a nanosized herbal medicine, are generated and combined with bathing water. When a user bathes in this bathtub water, the carbon dioxide nanobubbles or nanosized medical component contained in the bathtub water is absorbed into the user's skin, taken in capillary vessels and circulated around the body, allowing of obtaining a therapeutic effect and prevention effect for various illnesses.

For example, carbon dioxide nanobubbles and air nanobubbles can increase blood flow and improve blood circulation in a human body. In addition, while carbon dioxide can increase blood flow and improve blood circulation in a human body, it can increase an insulin-like growth factor, which is effective for various illnesses. Further, when nanosized medical component is absorbed into the user's skin and is taken in capillary vessels, the medical component is effectively functioned at an action site.

When a medical component is taken internally, a portion of it is decomposed in a digestive organ, the liver or the kidneys. However, such a medical component will not be decomposed through absorption of skin, allowing a less amount of the medical component to demonstrate its effect. Further, since bathing is a daily activity for a patient, it can be used as a therapeutic method and a prevention method with almost no antipathy.

Microbubbles are generated and sheared with a nanobubble generator having a gas-liquid shearing section, allowing nanobubbles to be certainly generated from carbon dioxide or air. Microbubbles are also generated by a microbubble generator and are sheared and pulverized by introduced into an ultra high-speed rotating section, allowing nanobubbles to be easily and certainly generated. Further, a microbubble generator can be used instead of a nano-liquid generator, and a microbubble generator can be used instead of a nanobubble generator. An economical system can be constructed with micro-liquid generator and microbubble generator since they can significantly reduce costs.

In addition, a micro-sized medical component are generated and sheared with a nano-liquid generator having a liquid shearing section, allowing the medical component to be easily and certainly nanosized.

Further, microbubbles are sheared by a nano-gas-liquid generator having a gas-liquid shearing section to generate nanobubbles and micro-liquid is sheared to generate a nanosized medical component easily and certainly.

A dissolved carbon dioxide analyzer is provided inside a bathtub and the amount of carbon dioxide introduced into a nanobubble generator is automatically controlled and adjusted using a electric-powered needle valve by a dissolved carbon dioxide controller according to a signal or signal level from the dissolved carbon dioxide analyzer, allowing a desired concentration of a dissolved carbon dioxide inside the bathtub to be set and a concentration of a dissolved carbon dioxide which a therapeutic effect and prevention effect can be obtained to be automatically set.

Further, a medical component tub can be filled with an herbal medicine, such as iris leaf, citrus, angelica root, chamomilla, cnidium rhizome, citrus unshiu peel, and ginseng, and a medical component extracted from the herbal medicine can be nanosized. Therefore, the nanosized medical component extracted from the herbal medicine can be absorbed through the skin, allowing of treating and preventing various illnesses. Further, the nanosized medical component extracted from the herbal medicine can increase blood flow, and the medical component that the herbal medicine contains can treat or prevent various illnesses. Prevention of the various illnesses means to increase an immune strength in order not to have any of such illnesses.

Further, a heater and a thermometer can be provided to control the temperature of the medical component tub using a temperature controller, allowing a medical component can be extracted in an appropriate extract condition from the herbal medicine that fills the medical component tub.

Various other modifications will be apparent to and can be readily made by those skilled in the art without departing from the scope and spirit of this invention. Accordingly, it is not intended that the scope of the claims appended hereto be limited to the description as set forth herein, but rather that the claims be broadly construed.

What is claimed is:

1. A bathtub apparatus, comprising: a nanobubble and/or nanosized medical component generating section to combine at least either of nanobubbles or a nanosized medical component with bath water from a bathtub and to circulate the bath water to the bathtub.

2. A bathtub apparatus according to claim 1, wherein the nanobubble and/or nanosized medical component generating section includes a gas-liquid mixture circulating pump for mixing and shearing bathtub water from the bathtub and gas from outside by a microbubble generating section to produce cloudy water full of microbubbles; and a gas-liquid shearing section for shearing microbubbles provided from the gas-liquid mixture circulating pump to generate nanobubbles.

3. A bathtub apparatus according to claim 1, wherein the nanobubble and/or nanosized medical component generating section includes a gas-liquid mixture circulating pump for mixing and shearing bathtub water from the bathtub and gas from outside by a microbubble generating section to produce cloudy water full of microbubbles; and a high speed rotating section, which is positioned inside the bathtub, for shearing and pulverizing microbubbles provided from the gas-liquid mixture circulating pump with difference between rotating speeds at a front and back of a discharge opening to generate nanobubbles.

4. A bathtub apparatus according to claim 1, wherein the nanobubble and/or nanosized medical component generating section includes a liquid-liquid mixture circulating pump for mixing and shearing bathtub water from the bathtub and a medical component liquid from outside by a micro-liquid generating section to produce cloudy water of micro-liquid; and a liquid shearing section for shearing a micro-liquid provided from the liquid-liquid mixture circulating pump to generate a nano-liquid.

5. A bathtub apparatus according to claim 1, wherein the nanobubble and/or nanosized medical component generating section includes a liquid-liquid mixture circulating pump for mixing and shearing bathtub water from the bathtub and a medical component liquid from outside by a micro-liquid generating section to produce cloudy water of micro-liquid; and a high speed rotating section, which is positioned inside the bathtub, for shearing and pulverizing a micro-liquid provided from the liquid-liquid mixture circulating pump with difference between rotating speeds at a front and back of a discharge opening to generate a nano-liquid.

6. A bathtub apparatus according to claim 1, wherein the nanobubble and/or nanosized medical component generating section includes a gas-liquid/liquid-liquid mixture circulating pump for mixing and shearing bathtub water from the bathtub and gas or a medical component liquid from outside by a microbubble/micro-liquid generating section to produce cloudy water full of microbubbles or a micro-liquid; and a gas-liquid/liquid-liquid shearing section for shearing microbubbles or a micro-liquid provided from the gas-liquid/liquid-liquid mixture circulating pump to generate nanobubbles or a nano-liquid.

7. A bathtub apparatus according to claim 1, wherein the nanobubble and/or nanosized medical component generating section includes a gas-liquid/liquid-liquid mixture circulating pump for mixing and shearing bathtub water from the bathtub and gas or a medical component liquid from outside by a microbubble/micro-liquid generating section to produce cloudy water full of microbubbles or a micro-liquid; and a high speed rotating section, which is positioned inside the bathtub, for shearing and pulverizing microbubbles or a micro-liquid provided from the gas-liquid/liquid-liquid mixture circulating pump with difference between rotating speeds at a front and back of a discharge opening to generate nanobubbles or a nano-liquid.

8. A bathtub apparatus according to claim 1, wherein the nanobubble and/or nanosized medical component generating section includes a gas-liquid mixture circulating pump for mixing and shearing bathtub water from the bathtub and gas from outside by a microbubble generating section to produce cloudy water full of microbubbles; a gas-liquid shearing section for shearing the microbubbles provided from the gas-liquid mixture circulating pump to generate nanobubbles; a liquid-liquid mixture circulating pump for mixing and shearing bathtub water from the bathtub and a medical component liquid from outside by a micro-liquid generating section to produce cloudy water of a micro-liquid; and a liquid shearing section for shearing a micro-liquid provided from the liquid-liquid mixture circulating pump to generate a nano-liquid.

9. A bathtub apparatus according to claim 1, wherein the nanobubble and/or nanosized medical component generating section includes a gas-liquid mixture circulating pump for mixing and shearing bathtub water from the bathtub and gas from outside by a microbubble generating section to produce cloudy water full of microbubbles; a first high speed rotating section, which is positioned inside the bathtub, for shearing and pulverizing microbubbles provided from the gas-liquid mixture circulating pump with difference between rotating speeds at a front and back of a discharge opening to generate nanobubbles; a liquid-liquid mixture circulating pump for mixing and shearing bathtub water from the bathtub and a medical component liquid from outside by a micro-liquid generating section to produce cloudy water of micro-liquid; and a second high speed shearing section, which is positioned inside the bathtub, for shearing and pulverizing a micro-liquid provided from the liquid-liquid mixture circulating pump with difference between rotating speeds at a front and back of a discharge opening to generate a nano-liquid.

10. A bathtub apparatus according to claim 2, wherein the gas is provided for the microbubble generating section via a needle valve.

11. A bathtub apparatus according to claim 3, wherein the gas is provided for the microbubble generating section via a needle valve.

12. A bathtub apparatus according to claim 6, wherein the gas is provided for the microbubble generating section via a needle valve.

13. A bathtub apparatus according to claim 7, wherein the gas is provided for the microbubble generating section via a needle valve.

14. A bathtub apparatus according to claim 8, wherein the gas is provided for the microbubble generating section via a needle valve.

15. A bathtub apparatus according to claim 9, wherein the gas is provided for the microbubble generating section via a needle valve.

16. A bathtub apparatus according to claim 1, wherein the nanobubbles are at least either of carbon dioxide nanobubbles or air nanobubbles.

17. A bathtub apparatus according to claim 2, wherein carbon dioxide as the gas is provided for the microbubble generating section from a liquefied carbon dioxide cylinder via a pressure-reducing valve and a needle valve.

18. A bathtub apparatus according to claim 3, wherein carbon dioxide as the gas is provided for the microbubble generating section from a liquefied carbon dioxide cylinder via a pressure-reducing valve and a needle valve.

19. A bathtub apparatus according to claim 6, wherein carbon dioxide as the gas is provided for the microbubble generating section from a liquefied carbon dioxide cylinder via a pressure-reducing valve and a needle valve.

20. A bathtub apparatus according to claim 7, wherein carbon dioxide as the gas is provided for the microbubble generating section from a liquefied carbon dioxide cylinder via a pressure-reducing valve and a needle valve.

21. A bathtub apparatus according to claim 8, wherein carbon dioxide as the gas is provided for the microbubble generating section from a liquefied carbon dioxide cylinder via a pressure-reducing valve and a needle valve.

22. A bathtub apparatus according to claim 9, wherein carbon dioxide as the gas is provided for the microbubble generating section from a liquefied carbon dioxide cylinder via a pressure-reducing valve and a needle valve.

23. A bathtub apparatus according to claim 3, wherein air as the gas is provided for the microbubble generating section via a valve.

24. A bathtub apparatus according to claim 6, wherein air as the gas is provided for the microbubble generating section via a valve.

25. A bathtub apparatus according to claim 7, wherein air as the gas is provided for the microbubble generating section via a valve.

26. A bathtub apparatus according to claim 8, wherein air as the gas is provided for the microbubble generating section via a valve.

27. A bathtub apparatus according to claim 9, wherein air as the gas is provided for the microbubble generating section via a valve.

28. A bathtub apparatus according to claim 1, wherein a plurality of nanobubble discharging sections, for which the nanobubbles are provided, are provided inside the bathtub, and a valve, whose degree of opening is independently adjustable, is provided for each of the plurality of the nanobubble discharging sections.

29. A bathtub apparatus according to claim 3, wherein a plurality of the high speed rotating sections are provided inside the bathtub, and a valve, whose degree of opening is independently adjustable, is provided for each of the plurality of the high speed rotating sections.

30. A bathtub apparatus according to claim 5, wherein a plurality of the high speed rotating sections are provided inside the bathtub, and a valve, whose degree of opening is independently adjustable, is provided for each of the plurality of the high speed rotating sections.

31. A bathtub apparatus according to claim 7, wherein a plurality of the high speed rotating sections are provided inside the bathtub, and a valve, whose degree of opening is independently adjustable, is provided for each of the plurality of the high speed rotating sections.

32. A bathtub apparatus according to claim 9, wherein a plurality of the high speed rotating sections are provided inside the bathtub, and a valve, whose degree of opening is independently adjustable, is provided for each of the plurality of the high speed rotating sections.

33. A bathtub apparatus according to claim 1, wherein a dissolved carbon dioxide analyzer is provided inside the bathtub and a signal input terminal of a dissolved carbon dioxide controller is electrically connected to a signal output terminal of the dissolved carbon dioxide analyzer, and the signal output terminal of the dissolved carbon dioxide controller is electrically connected to a control terminal of a needle valve for which carbon dioxide is provided, and a degree of opening of the needle valve is controlled by the dissolved carbon dioxide controller according to a concentration of dissolved carbon dioxide measured by the dissolved carbon dioxide analyzer, so that the amount of carbon dioxide introduced into the microbubble generating section from the needle valve is adjusted to a predetermined value.

34. A bathtub apparatus according to claim 4, further including a medical component tub that retains the medical component liquid and a medical component tub pump that is capable of sucking a medical component liquid from the medical component tub and providing the medical component liquid to the micro-liquid generating section.

35. A bathtub apparatus according to claim 5, further including a medical component tub that retains the medical component liquid and a medical component tub pump that is capable of sucking a medical component liquid from the medical component tub and providing the medical component liquid to the micro-liquid generating section.

36. A bathtub apparatus according to claim 6, further including a medical component tub that retains the medical component liquid and a medical component tub pump that is capable of sucking a medical component liquid from the medical component tub and providing the medical component liquid to the micro-liquid generating section.

37. A bathtub apparatus according to claim 7, further including a medical component tub that retains the medical component liquid and a medical component tub pump that is capable of sucking a medical component liquid from the medical component tub and providing the medical component liquid to the micro-liquid generating section.

38. A bathtub apparatus according to claim 8, further including a medical component tub that retains the medical component liquid and a medical component tub pump that is capable of sucking a medical component liquid from the medical component tub and providing the medical component liquid to the micro-liquid generating section.

39. A bathtub apparatus according to claim 9, further including a medical component tub that retains the medical component liquid and a medical component tub pump that is capable of sucking a medical component liquid from the medical component tub and providing the medical component liquid to the micro-liquid generating section.

40. A bathtub apparatus according to claim 34, further including a needle valve that is capable of controlling the amount of the medical component liquid between the medical component tub pump and the micro-liquid generating section.

41. A bathtub apparatus according to claim 35, further including a needle valve that is capable of controlling the amount of the medical component liquid between the medical component tub pump and the micro-liquid generating section.

42. A bathtub apparatus according to claim 36, further including a needle valve that is capable of controlling the amount of the medical component liquid between the medical component tub pump and the micro-liquid generating section.

43. A bathtub apparatus according to claim 37, further including a needle valve that is capable of controlling the amount of the medical component liquid between the medical component tub pump and the micro-liquid generating section.

44. A bathtub apparatus according to claim 38, further including a needle valve that is capable of controlling the amount of the medical component liquid between the medical component tub pump and the micro-liquid generating section.

45. A bathtub apparatus according to claim 39, further including a needle valve that is capable of controlling the amount of the medical component liquid between the medical component tub pump and the micro-liquid generating section.

46. A bathtub apparatus according to claim 34, wherein the medical component tub is filled with an herbal medicine and a medical component extracted from the herbal medicine is mixed with bathing water for use.

47. A bathtub apparatus according to claim 36, wherein the medical component tub is filled with an herbal medicine and a medical component extracted from the herbal medicine is mixed with bathing water for use.

48. A bathtub apparatus according to claim 37, wherein the medical component tub is filled with an herbal medicine and a medical component extracted from the herbal medicine is mixed with bathing water for use.

49. A bathtub apparatus according to claim 38, wherein the medical component tub is filled with an herbal medicine and a medical component extracted from the herbal medicine is mixed with bathing water for use.

50. A bathtub apparatus according to claim 39, wherein the medical component tub is filled with an herbal medicine and a medical component extracted from the herbal medicine is mixed with bathing water for use.

51. A bathtub apparatus according to claim 46, wherein one of iris leaf, citrus, angelica root, chamomilla, cnidium rhizome, citrus unshiu peel, ginseng, or two or more of the combination thereof is selected as the herbal medicine and fills the medical component tub.

52. A bathtub apparatus according to claim 47, wherein one of iris leaf, citrus, angelica root, chamomilla, cnidium rhizome, citrus unshiu peel, ginseng, or two or more of the combination thereof is selected as the herbal medicine and fills the medical component tub.

53. A bathtub apparatus according to claim 48, wherein one of iris leaf, citrus, angelica root, chamomilla, cnidium rhizome, citrus unshiu peel, ginseng, or two or more of the combination thereof is selected as the herbal medicine and fills the medical component tub.

54. A bathtub apparatus according to claim 49, wherein one of iris leaf, citrus, angelica root, chamomilla, cnidium rhizome, citrus unshiu peel, ginseng, or two or more of the combination thereof is selected as the herbal medicine and fills the medical component tub.

55. A bathtub apparatus according to claim 50, wherein one of iris leaf, citrus, angelica root, chamomilla, cnidium rhizome, citrus unshiu peel, ginseng, or two or more of the combination thereof is selected as the herbal medicine and fills the medical component tub.

56. A bathtub apparatus according to claim 34, wherein a heater, a thermometer and a temperature controller that is electrically connected to the thermometer are provided for the medical component tub, and the temperature controller is electrically connected to the heater, so that a temperature inside the medical component tub is controlled using the heater to a predetermined temperature by the temperature controller according to the temperature inside the medical component tub measured by the thermometer.

57. A bathtub apparatus according to claim 36, wherein a heater, a thermometer and a temperature controller that is electrically connected to the thermometer are provided for the medical component tub, and the temperature controller is electrically connected to the heater, so that a temperature inside the medical component tub is controlled using the heater to a predetermined temperature by the temperature controller according to the temperature inside the medical component tub measured by the thermometer.

58. A bathtub apparatus according to claim 37, wherein a heater, a thermometer and a temperature controller that is electrically connected to the thermometer are provided for the medical component tub, and the temperature controller is electrically connected to the heater, so that a temperature inside the medical component tub is controlled using the heater to a predetermined temperature by the temperature controller according to the temperature inside the medical component tub measured by the thermometer.

59. A bathtub apparatus according to claim 38, wherein a heater, a thermometer and a temperature controller that is electrically connected to the thermometer are provided for the medical component tub, and the temperature controller is electrically connected to the heater, so that a temperature inside the medical component tub is controlled using the heater to a predetermined temperature by the temperature controller according to the temperature inside the medical component tub measured by the thermometer.

60. A bathtub apparatus according to claim 39, wherein a heater, a thermometer and a temperature controller that is electrically connected to the thermometer are provided for the medical component tub, and the temperature controller is electrically connected to the heater, so that a temperature inside the medical component tub is controlled using the heater to a predetermined temperature by the temperature controller according to the temperature inside the medical component tub measured by the thermometer.

61. A bathtub apparatus according to claim 1, wherein the nanobubble and/or nanosized medical component generating section includes a gas-liquid mixture circulating pump for mixing and shearing bathtub water from the bathtub and gas from outside by a microbubble generating section to produce cloudy water full of microbubbles; and a gas-liquid shearing section for shearing microbubbles provided from the gas-liquid mixture circulating pump to generate nanobubbles; a liquid-liquid mixture circulating pump for mixing and shearing bathtub water from the bathtub and a medical component liquid from outside by a micro-liquid generating section to produce cloudy water of micro-liquid; and a second high speed rotating section, which is positioned inside the bathtub, for shearing and pulverizing a micro-liquid provided from the liquid-liquid mixture circulating pump with difference between rotating speeds at a front and back of a discharge opening to generate a nano-liquid.

62. A bathtub apparatus according to claim 1, wherein the nanobubble and/or nanosized medical component generating section includes a gas-liquid mixture circulating pump for mixing and shearing bathtub water from the bathtub and gas from outside by a microbubble generating section to produce cloudy water full of microbubbles; a first high speed rotating section, which is positioned inside the bathtub, for shearing and pulverizing microbubbles provided from the gas-liquid mixture circulating pump with difference between rotating speeds at a front and back of a discharge opening to generate nanobubbles; a liquid-liquid mixture circulating pump for mixing and shearing bathtub water from the bathtub and a medical component liquid from outside by a micro-liquid generating section to produce cloudy water of micro-liquid; and a liquid shearing section for shearing a micro-liquid provided from the liquid-liquid mixture circulating pump to generate a nano-liquid.

63. A therapeutic bathtub apparatus to use the bathtub apparatus according to claim 1 for treatment of various illnesses.

64. Bathing water in which either of nanobubbles or a nanosized medical component is combined with water.

65. Bathing water according to claim 64, wherein the nanobubbles are either of carbon dioxide nanobubbles or air nanobubbles.

66. Bathing water according to claim 64, including the nanobubbles generated by either shearing microbubbles or shearing and pulverizing the microbubbles with high speed rotation.

67. Bathing water according to claim 64, including the nanosized medical component generated by generating a micro-sized medical component and shearing the micro-sized medical component.

68. Bathing water according to claim 64, wherein the water is bathtub water and the nanobubbles are carbon dioxide nanobubbles, and at least either of the carbon dioxide nanobubbles or the nanosized medical component are combined with the bathtub water and circulated into the bathtub.

69. Bathing water according to claim 64, wherein the amount of the carbon dioxide nanobubbles generated is controlled, so that a concentration of carbon dioxide reaches a predetermined value based on a carbon dioxide concentration in bathtub water inside the bathtub and the carbon dioxide nanobubbles are circulated into the bathtub.

70. Therapeutic bathing water in which at least either of carbon dioxide nanobubbles or a nanosized medical component is combined with bathtub water.

71. Therapeutic bathing water according to claim 70, including at least either effect of absorbing either of the carbon dioxide nanobubbles or the nanosized medical component through the skin and taking it into a capillary vessel to increase blood flow and an insulin-like factor by the carbon dioxide nanobubbles; or circulating the nanosized medical component around a body to demonstrate a medicinal effect.
